(19) United States
(12) Patent Application Publication
(10) Pub. No.: US 2025/0269153 A1
(43) Pub. Date: Aug. 28, 2025

(54) BALLOON FOR BALLOON CATHETER, BALLOON CATHETER INCLUDING SAME, AND METHOD FOR PRODUCING BALLOON CATHETER

(71) Applicant: KANEKA CORPORATION, Osaka (JP)

(72) Inventors: Masahiro Kojima, Osaka (JP); Takahisa Hamabuchi, Osaka (JP); Yoshinori Nakano, Osaka (JP); Masato Tsueda, Osaka (JP)

(73) Assignee: KANEKA CORPORATION, Osaka (JP)

(21) Appl. No.: 19/207,011

(22) Filed: May 13, 2025

Related U.S. Application Data
(63) Continuation of application No. PCT/JP2023/040830, filed on Nov. 13, 2023.

(30) Foreign Application Priority Data
Nov. 16, 2022 (JP) 2022-183541
Nov. 16, 2022 (JP) 2022-183543

(51) Int. Cl. A61M 25/10

(52) U.S. Cl. CPC A61M 25/1029; A61M 2025/1075; A61M 2025/1086; A61M 2207/00

(57) ABSTRACT

A balloon for a balloon catheter having an outer layer, an inner layer composed of a material having a Shore D hardness lower than that of the outer layer and a protrusion part, is provided. A region where the protrusion part is present includes an outer layer protrusion part formed by the outer layer and protruding outward in the radial direction and an inner layer protrusion part formed by the inner layer and protruding outward in the radial direction. An angle (θ1) between a straight line that connects the two inner layer ends and a straight line that connects the inner layer apex and one of the two inner layer ends is smaller than an angle (θ2) between a straight line that connects the two outer layer ends and a straight line that connects the outer layer apex and one of the two outer layer ends.

### TECHNICAL FIELD

One or more embodiments of the present invention relate to a balloon for a balloon catheter, a balloon catheter including the same, and a method for producing a balloon catheter.

### BACKGROUND

Diseases such as angina pectoris and myocardial infarction are caused by the formation of stenotic areas hardened by calcification and other factors in inner walls of blood vessels. One of the treatments for these diseases is angioplasty, in which a balloon catheter is used to dilate the stenotic area. The angioplasty is a minimally invasive therapy that does not require an open chest procedure like bypass surgery and is widely used.

In the angioplasty, conventional balloon catheters may have difficulties in dilating stenotic areas hardened by calcification and other factors. Alternatively, a method is also used to expand the stenotic areas by placing a stent, a device that expands the stenotic areas, but in some cases, for example, after this treatment, a neointima of the blood vessel proliferates excessively and the blood vessel narrows again, causing an ISR (In-Stent-Restenosis) lesions may occur. The neointima in ISR lesions is soft and the surface is slippery, so a standard balloon catheter may cause the balloon to shift out of the lesion site during balloon dilation to damage the vessel.

As balloon catheters that can dilate a stenosis even in such calcified or ISR lesions, balloon catheters with a protrusion, blade or scoring element on the balloon to bite into the stenosis have been developed. For example, Patent document 1 discloses a balloon catheter in which an amorphous polymer is used for the protrusions, thereby increasing the stiffness of the protrusions relative to the balloon wall and improving the cutting efficiency of the protrusions.

### Patent Document

Patent document 1: US 2016/0128718 A1

However, the above conventional balloon catheters have a drawback in that the external shape of the protrusions may deform when the balloon, placed at the lesion site, is pressurized and expanded. Such deformation of the protrusions may reduce their ability to embed into the stenotic site, making it difficult to incise the stenosis, and may also cause the protrusions to unintentionally pierce areas of the vascular lumen wall.

The above conventional balloon catheters also have a drawback in that the protrusions provided at the leading end of the balloon, which becomes the foremost portion during advancement or retraction, may damage the inner wall of a vessel or other lumen when the deflated balloon is inserted into the lumen and delivered to the lesion site, or when it is withdrawn from the lesion site.

### SUMMARY

In view of the above circumstances, a balloon for a balloon catheter that is resistant to deformation of a protrusion part during balloon inflation, minimizes damage to the inner wall of a vessel or other lumen when inserted into the lumen, and improves both deliverability within the lumen and incision efficiency at the stenotic site, is provided. A balloon catheter equipped with such a balloon, as well as a method for manufacturing the balloon catheter are also provided.

The first balloon for a balloon catheter according to one or more embodiments of the present invention, which has been made to address the above, is as follows.
[1] A balloon for a balloon catheter having a longitudinal axis direction, a radial direction, and a circumferential direction, comprising:
an outer layer; and
an inner layer composed of a material having a Shore D hardness lower than that of the outer layer,
the balloon comprising:
a straight tubular part;
a proximal tapered part located proximal to the straight tubular part;
a proximal sleeve part located proximal to the proximal tapered part;
a distal tapered part located distal to the straight tubular part; and
a distal sleeve part located distal to the distal tapered part,
the balloon further comprising a protrusion part that protrudes outward in the radial direction and extends in the longitudinal axis direction, wherein
in a cross section perpendicular to the longitudinal axis direction at the straight tubular part, a region where the protrusion part is present includes:
an outer layer protrusion part formed by the outer layer and protruding outward in the radial direction; and
an inner layer protrusion part formed by the inner layer and protruding outward in the radial direction,
the outer layer protrusion part includes:
an outer layer apex being an apex of the outer layer protrusion part; and
outer layer ends located on both sides in the circumferential direction at respective circumferential ends of the outer layer protrusion part,
the inner layer protrusion part includes:
an inner layer apex being an apex of the inner layer protrusion part; and
inner layer ends located on both sides in the circumferential direction at respective circumferential ends of the inner layer protrusion part, and
in the cross section perpendicular to the longitudinal axis direction at the straight tubular part, an angle formed in a first direction in the circumferential direction between a straight line that connects the two inner layer ends and a straight line that connects the inner layer apex and one of the two inner layer ends is smaller than an angle formed in the first direction in the circumferential direction between a straight line that connects the two outer layer ends and a straight line that connects the outer layer apex and one of the two outer layer ends.
[2] The balloon for a balloon catheter according to [1], wherein, in a cross section perpendicular to the longitudinal axis direction at the straight tubular part, the inner layer apex is located further outward in the radial direction than a straight line that connects the two outer layer ends.
[3] The balloon for a balloon catheter according to [1] or [2], wherein, in a cross section perpendicular to the longitudinal axis direction at the straight tubular part, an angle at the inner layer apex in a triangle formed by connecting the two inner layer ends and the inner layer apex is an obtuse angle, and, in the cross section perpendicular to the longitudinal axis direction at the straight tubular part, an angle at the outer layer apex in a triangle formed by connecting the two outer layer ends and the outer layer apex is an acute angle.
[4] The balloon for a balloon catheter according to any one of [1] to [3], wherein, in a cross section perpendicular to the longitudinal axis direction at the straight tubular part, an area of the inner layer protrusion part is smaller than an area of the outer layer protrusion part.
[5] The balloon for a balloon catheter according to any one of [1] to [4], wherein, in a cross section perpendicular to the longitudinal axis direction at at least one of the proximal tapered part and the distal tapered part, an angle formed in the first direction in the circumferential direction between a straight line that connects the two inner layer ends and a straight line that connects the inner layer apex and one of the two inner layer ends is smaller than an angle formed in the first direction in the circumferential direction between a straight line that connects the two outer layer ends and a straight line that connects the outer layer apex and one of the two outer layer ends.
[6] The balloon for a balloon catheter according to any one of [1] to [5], wherein, in a cross section perpendicular to the longitudinal axis direction at at least one of the proximal sleeve part and the distal sleeve part, an angle formed in the first direction in the circumferential direction between a straight line that connects the two inner layer ends and a straight line that connects the inner layer apex and one of the two inner layer ends is greater than an angle formed in the first direction in the circumferential direction between a straight line that connects the two outer layer ends and a straight line that connects the outer layer apex and one of the two outer layer ends.
[7] The balloon for a balloon catheter according to any one of [1] to [5], wherein, in a cross section perpendicular to the longitudinal axis direction at at least one of the proximal sleeve part and the distal sleeve part, an angle formed in the first direction in the circumferential direction between a straight line that connects the two inner layer ends and a straight line that connects the inner layer apex and one of the two inner layer ends is smaller than an angle formed in the first direction in the circumferential direction between a straight line that connects the two outer layer ends and a straight line that connects the outer layer apex and one of the two outer layer ends.

One or more embodiments of the present invention also provide a balloon catheter including the first balloon for a balloon catheter. A first balloon catheter according to one or more embodiments of the present invention is as follows.
[8] A balloon catheter comprising the balloon for a balloon catheter according to any one of [1] to [7].

One or more embodiments of the present invention further provide a method for producing the balloon catheter according to [8]. A method for producing a first balloon catheter according to one or more embodiments of the present invention is as follows.
[9] A method for producing the balloon catheter according to [8], the method comprising:
a step of preparing a parison having a radial direction, a circumferential direction, and a longitudinal axis direction, the parison having a lumen extending in the longitudinal axis direction, and
a step of stretching the parison to produce a balloon including the proximal sleeve part, the proximal tapered part, the straight tubular part, the distal tapered part, and the distal sleeve part, the balloon including the protrusion part protruding outward in the radial direction and extending in the longitudinal axis direction, wherein
the parison includes:
an outer layer, and an inner layer composed of a material having a Shore D hardness lower than that of the outer layer; and
a protruding region including the protrusion part that protrudes outward in the radial direction and extends in the longitudinal axis direction, and a non-protruding region other than the protruding region, wherein
in a cross section perpendicular to the longitudinal axis direction, the inner layer includes a thin portion in the non-protruding region and a thick portion in the protruding region, the thick portion having a thickness greater than a thickness of the thin portion.

The second balloon for a balloon catheter according to one or more embodiments of the present invention, which has been made to solve the above-described issues, is as follows.
[10] A balloon for a balloon catheter having a longitudinal axis direction, a radial direction, and a circumferential direction, comprising:
an outer layer; and
an inner layer composed of a material having a Shore D hardness lower than that of the outer layer,
the balloon comprising:
a straight tubular part;
a proximal tapered part located proximal to the straight tubular part;
a proximal sleeve part located proximal to the proximal tapered part;
a distal tapered part located distal to the straight tubular part; and
a distal sleeve part located distal to the distal tapered part,
the balloon further comprising a protrusion part that protrudes outward in the radial direction and extends in the longitudinal axis direction, wherein
in a cross section perpendicular to the longitudinal axis direction at the straight tubular part, a region where the protrusion part is present includes:
an outer layer protrusion part formed by the outer layer and protruding outward in the radial direction; and
an inner layer protrusion part formed by the inner layer and protruding outward in the radial direction,
the outer layer protrusion part includes:
an outer layer apex being an apex of the outer layer protrusion part; and
outer layer ends located on both sides in the circumferential direction at respective circumferential ends of the outer layer protrusion part,
the inner layer protrusion part includes:
an inner layer apex being an apex of the inner layer protrusion part; and
inner layer ends located on both sides in the circumferential direction at respective circumferential ends of the inner layer protrusion part, and
a ratio (θ2/θ1) of an angle θ2 formed in the first direction in the circumferential direction between a straight line that connects the two outer layer ends and a straight line that connects the outer layer apex and one of the two outer layer ends to an angle θ1 formed in the first direction in the circumferential direction between a straight line that connects the two inner layer ends and a straight line that connects the inner layer apex and one of the two inner layer ends, in the cross section perpendicular to the longitudinal axis direction at the straight tubular part, is greater than a ratio (θ4/θ3) of an angle θ4 formed in the first direction in the circumferential direction between a straight line that connects the two outer layer ends and a straight line that connects the outer layer apex and one of the two outer layer ends to an angle θ3 formed in the first direction in the circumferential direction between a straight line that connects the two inner layer ends and a straight line that connects the inner layer apex and one of the two inner layer ends, in a cross section perpendicular to the longitudinal axis direction at at least one of the proximal tapered part and the distal tapered part.

[11] The balloon for a balloon catheter according to [10], wherein a ratio (θ2/θ1) of an angle θ2 formed in the first direction in the circumferential direction between a straight line that connects the two outer layer ends and a straight line that connects the outer layer apex and one of the two outer layer ends to an angle θ1 formed in the first direction in the circumferential direction between a straight line that connects the two inner layer ends and a straight line that connects the inner layer apex and one of the two inner layer ends, in a cross section perpendicular to the longitudinal axis direction at the straight tubular part, is greater than a ratio (θ6/θ5) of an angle θ6 formed in the first direction in the circumferential direction between a straight line that connects the two outer layer ends and a straight line that connects the outer layer apex and one of the two outer layer ends to an angle θ5 formed in the first direction in the circumferential direction between a straight line that connects the two inner layer ends and a straight line that connects the inner layer apex and one of the two inner layer ends, in a cross section perpendicular to the longitudinal axis direction at at least one of the proximal sleeve part and the distal sleeve part.
[12] The balloon for a balloon catheter according to [10] or [11], wherein a ratio (θ4/θ3) of an angle θ4 formed in the first direction in the circumferential direction between a straight line that connects the two outer layer ends and a straight line that connects the outer layer apex and one of the two outer layer ends to an angle θ3 formed in the first direction in the circumferential direction between a straight line that connects the two inner layer ends and a straight line that connects the inner layer apex and one of the two inner layer ends, in a cross section perpendicular to the longitudinal axis direction at at least one of the proximal tapered part and the distal tapered part, is greater than a ratio (θ6/θ5) of an angle θ6 formed in the first direction in the circumferential direction between a straight line that connects the two outer layer ends and a straight line that connects the outer layer apex and one of the two outer layer ends to an angle θ5 formed in the first direction in the circumferential direction between a straight line that connects the two inner layer ends and a straight line that connects the inner layer apex and one of the two inner layer ends, in a cross section perpendicular to the longitudinal axis direction at at least one of the proximal sleeve part and the distal sleeve part.
[13] The balloon for a balloon catheter according to any one of [10] to [12], wherein an angle at the inner layer apex formed in a triangle defined by connecting the two inner layer ends and the inner layer apex, in a cross section perpendicular to the longitudinal axis direction at the straight tubular part, is greater than an angle at the inner layer apex formed in a triangle defined by connecting the two inner layer ends and the inner layer apex, in a cross section perpendicular to the longitudinal axis direction at at least one of the proximal tapered part and the distal tapered part.
[14] The balloon for a balloon catheter according to any one of [10] to [13], wherein a proportion of an area of the inner layer in the protrusion part in a cross section perpendicular to the longitudinal axis direction at the straight tubular part is smaller than a proportion of an area of the inner layer in the protrusion part in a cross section perpendicular to the longitudinal axis direction at at least one of the proximal tapered part and the distal tapered part.

One or more embodiments of the present invention also provide a balloon catheter including the second balloon for a balloon catheter. A second balloon catheter according to one or more embodiments of the present invention is as follows.
[15] A balloon catheter comprising the balloon for a balloon catheter according to any one of [10] to [14].

One or more embodiments of the present invention further provide a method for producing the balloon catheter according to [15]. A method for producing a second balloon catheter according to one or more embodiments of the present invention is as follows.
[16] A method for producing the balloon catheter according to [15], the method comprising:
a step of preparing a parison having a radial direction, a circumferential direction, and a longitudinal axis direction, the parison having a lumen extending in the longitudinal axis direction; and
a step of stretching the parison to produce a balloon including the proximal sleeve part, the proximal tapered part, the straight tubular part, the distal tapered part, and the distal sleeve part, the balloon including the protrusion part protruding outward in the radial direction and extending in the longitudinal axis direction, wherein
the parison includes:
an outer layer, and an inner layer composed of a material having a Shore D hardness lower than that of the outer layer; and
a protruding region including the protrusion part that protrudes outward in the radial direction and extends in the longitudinal axis direction, and a non-protruding region other than the protruding region, wherein
in a cross section perpendicular to the longitudinal axis direction, the inner layer includes a thin portion in the non-protruding region and a thick portion in the protruding region, the thick portion having a thickness greater than a thickness of the thin portion.

According to the above-described first and second balloons for a balloon catheter, the balloon catheters including the same, and the methods for producing the balloon catheters, it is possible to provide a balloon for a balloon catheter, a balloon catheter including the same, and a method for producing the balloon catheter, in which the shape of the protrusion part is less likely to deform when the balloon is inflated, and the inner wall of a lumen such as a blood vessel is less likely to be damaged when the balloon is inserted into the lumen, thereby improving the deliverability in the lumen and the efficiency of incision of the stenosis. As a result, it becomes possible to safely and efficiently incise a stenosis during treatment or procedures using the balloon catheter.

### BRIEF DESCRIPTION OF THE DRAWINGS

FIG. 1 is a side view of a balloon catheter according to one or more embodiments of the present invention.

FIG. 2 is a cross-sectional view taken along line II-II of the balloon catheter shown in FIG. 1.

FIG. 3 is a cross-sectional view taken along line III-III of the balloon catheter shown in FIG. 1.

FIG. 4 is a cross-sectional view taken along line IV-IV of the balloon catheter shown in FIG. 1.

FIG. 5 is a perspective view of a parison before stretching according to one or more embodiments of the present invention. FIG. 6 is a cross-sectional view taken along line VI-VI of the parison shown in FIG. 5.

FIG. 7 is a cross-sectional view perpendicular to the longitudinal axis direction of a parison mold used for producing the parison shown in FIG. 6.

FIG. 8 is a longitudinal cross-sectional view of a mold used for stretching the parison in the production method according to one or more embodiments of the present invention.

FIG. 9 is a cross-sectional view taken along line IX-IX of the mold shown in FIG. 8.

### DETAILED DESCRIPTION

Hereinafter, one or more embodiments of the present invention will be described based on the following embodiments, however, the present invention is not limited by the following embodiments and can be altered in design within a scope in compliance with the intent described above and below, and all the changes are to be encompassed within a technical scope of the present invention. Note that, in each drawing, hatching, reference signs for components, and the like may be omitted for convenience of description, and in such a case, the specification and other drawings are to be referred to. Furthermore, since the dimensions of the various components in the drawings are provided for the purpose of facilitating the understanding of the feature of one or more embodiments of the present invention, the dimensions may differ from the actual dimensions in some cases.

### 1. Balloon for Balloon Catheter

First, a description will be given of a first balloon for a balloon catheter. A first balloon for a balloon catheter in accordance with one or more embodiments of the present invention has a longitudinal axis direction, a radial direction, and a circumferential direction, and has an outer layer; and an inner layer composed of a material having a Shore D hardness lower than that of the outer layer; the balloon has a straight tubular part, a proximal tapered part located proximal to the straight tubular part, a proximal sleeve part located proximal to the proximal tapered part, a distal tapered part located distal to the straight tubular part, and a distal sleeve part located distal to the distal tapered part; the balloon further has a protrusion part that protrudes outward in the radial direction and extends in the longitudinal axis direction, wherein, in a cross section perpendicular to the longitudinal axis direction at the straight tubular part, a region where the protrusion part is present includes an outer layer protrusion part formed by the outer layer and protruding outward in the radial direction and an inner layer protrusion part formed by the inner layer and protruding outward in the radial direction; the outer layer protrusion part includes an outer layer apex being an apex of the outer layer protrusion part, and outer layer ends located on both sides in the circumferential direction at respective circumferential ends of the outer layer protrusion part; the inner layer protrusion part includes an inner layer apex being an apex of the inner layer protrusion part, and inner layer ends located on both sides in the circumferential direction at respective circumferential ends of the inner layer protrusion part; and, in the cross section perpendicular to the longitudinal axis direction at the straight tubular part, an angle formed in a first direction in the circumferential direction between a straight line that connects the two inner layer ends and a straight line that connects the inner layer apex and one of the two inner layer ends is smaller than an angle formed in the first direction in the circumferential direction between a straight line that connects the two outer layer ends and a straight line that connects the outer layer apex and one of the two outer layer ends.

The dilation of a stenosis using a balloon catheter is performed by inserting a balloon provided at a distal end of the balloon catheter into a vascular lumen, delivering it to the stenosis, inflating the balloon, and incising the stenosis by causing a protrusion part provided outward in the radial direction of the balloon to dig into the stenosis. According to the above-described balloon for a balloon catheter, which includes an outer layer and an inner layer composed of a material having a Shore D hardness lower than that of the outer layer, an angle formed in a first direction in the circumferential direction between a straight line connecting the two inner layer ends and a straight line connecting the inner layer apex and one of the inner layer ends is smaller than an angle formed in the same direction between a straight line connecting the two outer layer ends and a straight line connecting the outer layer apex and one of the outer layer ends, whereby, when the balloon is pressurized for inflation, the inner layer in the protrusion part tends to stretch more easily than the outer layer in the circumferential direction. As the inner layer preferentially stretches in the circumferential direction within the protrusion part, the circumferential elongation of the outer layer is suppressed, making the shape of the protrusion part less likely to deform. This allows for efficient incision of the stenosis while improving the safety of treatment or procedures using the balloon catheter.

When the balloon is inserted into the stenosis or withdrawn from the body, the fluid inside the lumen of the balloon is discharged to deflate the balloon, thereby reducing the outer diameter of the balloon by wrapping the wing-shaped portion of the balloon around the shaft of the balloon catheter. At this time, the protrusion part provided on the inflatable portion of the balloon is covered by the wing-shaped portion, which can prevent damage caused by the protrusion part coming into contact with the inner wall of the blood vessel.

In the present specification, the balloon for a balloon catheter may sometimes be simply referred to as “the balloon.”

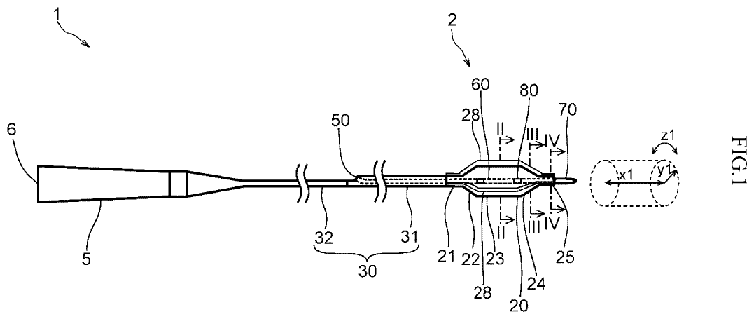

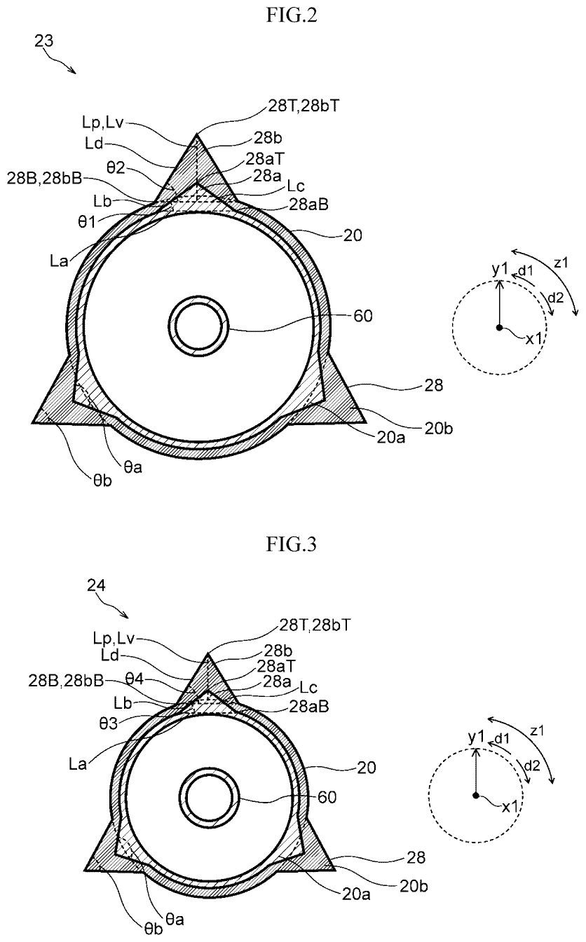

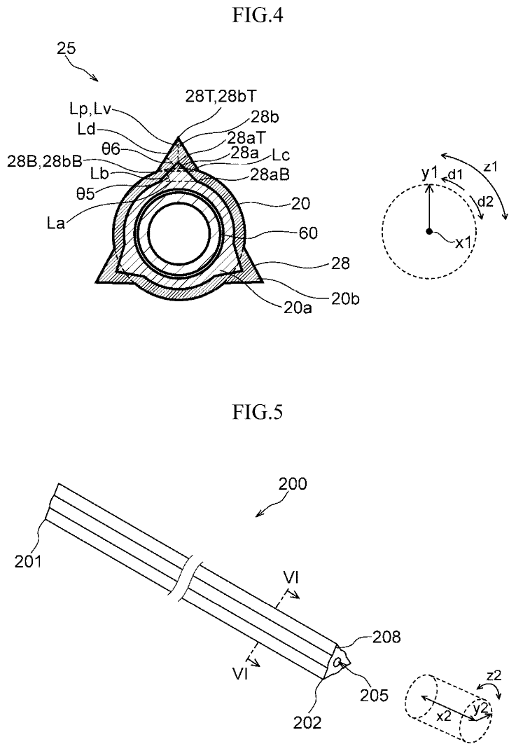

Hereinafter, a balloon for a balloon catheter according to one or more embodiments of the present invention will be described with reference to FIGS. 1 to 4. FIG. 1 is a side view of a balloon catheter according to one or more embodiments of the present invention. FIG. 2 is a cross-sectional view taken along line II-II of the balloon catheter shown in FIG. 1, and shows a cross section perpendicular to the longitudinal axis direction at the straight tubular part. FIG. 3 is a cross-sectional view taken along line III-III of the balloon catheter shown in FIG. 1, and shows a cross section perpendicular to the longitudinal axis direction at the distal tapered part. FIG. 4 is a cross-sectional view taken along line IV-IV of the balloon catheter shown in FIG. 1, and shows a cross section perpendicular to the longitudinal axis direction at the distal sleeve part.

As shown in FIG. 1, a balloon 2 is used in a balloon catheter 1. The balloon 2 is connected to the distal end part of a shaft 30, and the balloon 2 can be inflated by introducing a fluid through the lumen of the shaft 30 and deflated by discharging the fluid. To control the inflation and deflation of the balloon 2, a fluid can be introduced or discharged using an indeflator (a balloon pressurizer). The fluid may be a pressurized fluid supplied by a pump or the like. Details of the balloon catheter 1 will be described in Section “2. Balloon catheter.”

The balloon 2 has a longitudinal axis direction x1, a radial direction y1 that connects the figure center of the outer edge of the balloon 2 and a point on the outer edge in a cross section perpendicular to the longitudinal axis direction x1, and a circumferential direction z1 along the outer edge in a cross section perpendicular to the longitudinal axis direction x1. In the present specification, the direction toward the operator along the longitudinal axis direction x1 is referred to as the proximal side, and the direction opposite to the proximal side, that is, the direction toward the patient, is referred to as the distal side.

Components or portions other than the balloon 2 each have a longitudinal axis direction, a radial direction, and a circumferential direction, which may or may not be the same as the longitudinal axis direction x1, the radial direction y1, and the circumferential direction z1 of the balloon 2. However, in the present specification, for ease of understanding, all components and portions are described as having the same longitudinal axis direction, radial direction, and circumferential direction as the longitudinal axis direction x1, radial direction y1, and circumferential direction z1 of the balloon 2.

As shown in FIGS. 1 to 4, the balloon 2 includes a protrusion part 28 that protrudes outward in the radial direction y1 and extends in the longitudinal axis direction x1. The protrusion part 28 is a portion that is formed to be thicker than a portion of the balloon 2 where no protrusion part 28 is provided. In other words, as shown in FIGS. 2 to 4, the protrusion part 28 can also be described as a portion that protrudes outward in the radial direction y1 from the outer surface of a balloon body part 20, which has a thickness corresponding to a portion where no protrusion part 28 is provided.

The thickness of the protrusion part 28 of the balloon 2 may be 1.2 times or more, 1.5 times or more, 1.8 times or more, 2.0 times or more, or 2.5 times or more the thickness of a portion of the balloon 2 where no protrusion part 28 is provided. The upper limit of the thickness of the protrusion part 28 of the balloon 2 is not particularly limited, and for example, it may be 30 times or less, 20 times or less, or 10 times or less the thickness of a portion where no protrusion part 28 is provided.

The balloon body part 20 defines the basic shape of the balloon 2, and the protrusion part 28 may be provided on the outer surface of the balloon body part 20 in an arbitrary pattern such as a linear, dot-like, mesh-like, or spiral pattern. The protrusion part 28 provides the balloon 2 with a scoring function, enabling the balloon 2 to create cracks in a calcified stenosis and dilate it during angioplasty. The protrusion part 28 can also contribute to improving the strength of the balloon 2 and suppressing overexpansion during pressurization.

As shown in FIGS. 2 to 4, a plurality of protrusion parts 28 may be provided in the circumferential direction z1, or only one protrusion part 28 may be provided. The number of protrusion parts 28 in the circumferential direction z1 may be 1 or more, 2 or more, 3 or more, 4 or more, or 6 or more, and may also be 20 or less, 15 or less, or 10 or less. When a plurality of protrusion parts 28 are provided in the circumferential direction z1, the plurality of protrusion parts 28 may be spaced apart from each other in the circumferential direction z1. They may be arranged at equal intervals in the circumferential direction z1. The spacing between the plurality of protrusion parts 28 may be greater than the maximum circumferential length of each protrusion part 28.

The cross-sectional shape of the protrusion part 28 in a cross section perpendicular to the longitudinal axis direction x1 may be of any shape. For example, it may be triangular, quadrilateral, polygonal, semicircular, a part of a circle, substantially circular, fan-shaped, wedge-shaped, convex-shaped, spindle-shaped, or a combination thereof. It should be noted that triangular, quadrilateral, and polygonal shapes include not only those with sharp corners and straight edges, but also so-called rounded polygons in which the corners are rounded, and those in which at least part of the edge is curved. Alternatively, the cross-sectional shape of the protrusion part 28 may be an irregular shape having irregularities, notches, or the like. When the protrusion part 28 is formed in a linear or dot-like shape, the protrusion part 28 may be arranged so as to extend along the longitudinal axis direction x1. Alternatively, the protrusion part 28 may be arranged so as to extend in a helical shape around the longitudinal axis.

Although not shown in the figures, the balloon 2 may include an inner protrusion part that protrudes inward in the radial direction y1. The inner protrusion part may extend in the longitudinal axis direction x1. The protrusion part 28 and the inner protrusion part may be arranged at the same position in the longitudinal axis direction x1 or the circumferential direction z1 of the balloon 2, and they may be integrally formed. The balloon 2 may include the protrusion part 28 and the inner protrusion part, as the protrusion part 28, the balloon body part 20, and the inner protrusion part are integrally formed to have an increased thickness.

The balloon 2 includes an outer layer 20b and an inner layer 20a that is located inward in the radial direction relative to the outer layer 20b and is composed of a material having a Shore D hardness lower than that of the outer layer 20b. The balloon 2 may have a two-layer structure composed of the inner layer 20a and the outer layer 20b in all portions.

The inner layer 20a and the outer layer 20b may be continuously present over the entire 360 degrees in the circumferential direction z1 at any position in the longitudinal axis direction x1. The balloon 2 having a two-layer structure composed of the inner layer 20a and the outer layer 20b in all portions has an outer surface formed from the outer layer 20b having a high Shore D hardness, which makes the outer surface resistant to damage and improves its strength. The outer surface of the protrusion part 28 is also formed from the outer layer 20b having a high Shore D hardness, thereby enhancing the scoring function of the protrusion part 28.

The Shore D hardness of the inner layer 20a may be 20 or more, 25 or more, 30 or more, 35 or more, or 40 or more, and may be 70 or less, 65 or less, 60 or less, or 55 or less. The Shore D hardness of the outer layer 20b may be more than 70, 72 or more, 74 or more, or 75 or more, and may be 90 or less, 85 or less, or 80 or less. When the Shore D hardness of the inner layer 20a is within the above range, it can contribute to improving the flexibility of the balloon 2. When the Shore D hardness of the outer layer 20b is within the above range, it can contribute to improving the strength of the balloon 2 and enhancing the scoring function of the protrusion part 28.

The Shore D hardness can be measured, for example, using a Type D durometer based on JIS K6253-2:2012. The respective Shore D hardness values of the inner layer 20a and the outer layer 20b may be those of the materials in the stage before being formed into the balloon 2.

As the material for the outer layer 20b, polyamide resins such as nylon 11 and nylon 12; polyester resins such as polyethylene terephthalate and polybutylene terephthalate; and polyurethane resins may be used. As the material for the inner layer 20a, from the viewpoint of having a lower Shore D hardness, it may be preferable to use a thermoplastic elastomer, and for example, polyamide elastomers such as polyether block amide copolymers may be used.

As shown in FIG. 1, the balloon 2 has a proximal end and a distal end in the longitudinal axis direction x1, and includes a straight tubular part 23, a proximal tapered part 22 located proximal to the straight tubular part 23, a proximal sleeve part 21 located proximal to the proximal tapered part 22, a distal tapered part 24 located distal to the straight tubular part 23, and a distal sleeve part 25 located distal to the distal tapered part 24. The straight tubular part 23 may be substantially cylindrical and have approximately the same diameter in the longitudinal axis direction x1, but it may have different diameters in the longitudinal axis direction x1. The proximal tapered part 22 and the distal tapered part 24 may be formed in a substantially conical or frustoconical shape, tapering toward the proximal end and the distal end, respectively, away from the straight tubular part 23. When the straight tubular part 23 has the maximum diameter, it can easily make sufficient contact with a lesion such as a stenosis when the balloon 2 is inflated at the lesion site, thereby facilitating treatment such as dilation of the lesion. In addition, because the proximal tapered part 22 and the distal tapered part 24 are tapered, the outer diameters of the proximal and distal ends of the balloon 2 can be reduced when the balloon 2 is deflated, thereby reducing the step between the shaft 30 and the balloon 2 and improving the ease of insertion of the balloon 2 into a body lumen.

While the proximal tapered part 22, the straight tubular part 23, and the distal tapered part 24 are portions that expand when fluid is introduced into the balloon 2, the proximal sleeve part 21 and the distal sleeve part 25 may not expand. This allows at least a part of the proximal sleeve part 21 to be fixed to the distal end of the shaft 30, and at least a part of the distal sleeve part 25 to be fixed to an inner shaft 60, which will be described later.

The balloon 2 may include the protrusion part 28 in each of the regions: the proximal sleeve part 21, the proximal tapered part 22, the straight tubular part 23, the distal tapered part 24, and the distal sleeve part 25. This allows the protrusion part 28 provided in the straight tubular part 23 to contribute to enhancing the scoring function, while the protrusion parts 28 provided in regions other than the straight tubular part 23 can contribute to improving the strength of the balloon 2 and suppressing overexpansion during pressurization.

As shown in FIG. 2, in the straight tubular part 23, the protrusion part 28 includes an apex 28T, which is an outer end in the radial direction y1, and base ends 28B, which is located inward of the apex 28T in the radial direction y1 and is connected to the outer surface of the balloon 2. The protrusion part 28 having the apex 28T can facilitate incision of the stenosis and improve the incision efficiency by the protrusion part 28. As shown in FIGS. 3 and 4, the protrusion part 28 in the proximal sleeve part 21, the proximal tapered part 22, the distal tapered part 24, and the distal sleeve part 25 may also include the apex 28T.

When the position of the apex 28T is difficult to define due to processing that deforms or removes the outermost tip portion in the radial direction y1 of the protrusion part 28, the apex 28T may be defined as the point where a contour line of the outer shape of the protrusion part 28 intersects a straight line passing through a midpoint in the width direction of the base ends 28B and a figure center of the outer profile of the balloon 2 in a cross section perpendicular to the longitudinal axis direction x1. The midpoint in the width direction of the base ends 28B refers to a midpoint of a line segment connecting an end on the first direction d1 side and an end on the second direction d2 side in the circumferential direction z1 of the protrusion part 28.

The protrusion part 28 provided in the straight tubular part 23 may be tilted in either the first direction d1 or the second direction d2 in the circumferential direction z1. The angle at which the protrusion part 28 provided in the straight tubular part 23 is tilted in either the first direction d1 or the second direction d2 in the circumferential direction z1 may be within a predetermined range. This makes it possible to efficiently perform fixation of the balloon 2 to the lesion site and incision of the stenosis by the protrusion part 28. When the protrusion part 28 is tilted in either the first direction d1 or the second direction d2 in the circumferential direction z1, it may be preferable that a straight line Lp, which connects a midpoint in the width direction of the base ends 28B and the apex 28T, substantially coincides with the perpendicular line Lv of the base ends 28B, that is, the angle formed between the straight line Lp and the perpendicular line Lv of the base ends 28B is close to 0 degrees. The absolute value of the angle may be 5 degrees or less, 10 degrees or less, or 15 degrees or less. In this case, the angle formed between the straight line Lp and the perpendicular line Lv of the base ends 28B is defined as an angle formed in a direction in which the protrusion part 28 is tilted with respect to the perpendicular line Lv of the base ends 28B, with a midpoint in the width direction of the base ends 28B serving as a starting point. Here, the perpendicular line Lv of the base ends 28B is defined as a line perpendicular to a line segment connecting one end and the other end of the base ends 28B in the circumferential direction z1, drawn from the apex 28T in a cross section in the radial direction y. The midpoint in the width direction of the base ends 28B refers to a midpoint of a line segment connecting an end on the first direction d1 side and an end on the second direction d2 side in the circumferential direction z1 of the protrusion part 28.

As shown in FIG. 2, in a cross section perpendicular to the longitudinal axis direction x1 at the straight tubular part 23, a region where the protrusion part 28 is present includes an outer layer protrusion part 28b, which is formed by the outer layer 20b and protrudes outward in the radial direction y1, and an inner layer protrusion part 28a, which is formed by the inner layer 20a and protrudes outward in the radial direction y1.

The outer layer protrusion part 28b includes an outer layer apex 28bT, which is the apex of the outer layer protrusion part 28b, and outer layer ends 28bB, which are located on both sides in the circumferential direction z1 of the outer layer apex 28bT and at respective circumferential ends of the outer layer protrusion part 28b. The inner layer protrusion part 28a includes an inner layer apex 28aT, which is the apex of the inner layer protrusion part 28a, and inner layer ends 28aB, which are located on both sides in the circumferential direction z1 of the inner layer apex 28aT and at respective circumferential ends of the inner layer protrusion part 28a. That is, the outer layer protrusion part 28b has two outer layer ends 28bB in the circumferential direction z1, with the outer layer apex 28bT located between the two outer layer ends 28bB, and the inner layer protrusion part 28a has two inner layer ends 28aB in the circumferential direction z1, with the inner layer apex 28aT located between the two inner layer ends 28aB.

When it is difficult to define the position of the outer layer apex 28bT due to processing such as deformation or removal of a tip portion on the outer side in the radial direction y1 of the protrusion part 28, the outer layer apex 28bT may be defined as a point where a straight line connecting a midpoint of a line segment between the two outer layer ends 28bB and a figure center of the outer contour of the balloon 2 intersects with an outline of the outer layer protrusion part 28b in a cross section perpendicular to the longitudinal axis direction x1. Similarly, when it is difficult to define the position of the inner layer apex 28aT, the inner layer apex 28aT may be defined as a point where a straight line connecting a midpoint of a line segment between the two inner layer ends 28aB and the figure center of the outer contour of the balloon 2 intersects with an outline of the inner layer protrusion part 28a in a cross section perpendicular to the longitudinal axis direction x1.

As shown in FIG. 2, in a cross section perpendicular to the longitudinal axis direction x1 at the straight tubular part 23, an angle θ1 formed in the first direction d1 in the circumferential direction z1 between a straight line La that connects the two inner layer ends 28aB and a straight line Lb that connects the inner layer apex 28aT and one of the two inner layer ends 28aB is smaller than an angle θ2 formed in the first direction d1 in the circumferential direction z1 between a straight line Lc that connects the two outer layer ends 28bB and a straight line Ld that connects the outer layer apex 28bT and one of the two outer layer ends 28bB.

In the straight tubular part 23, since the angle θ1 formed between the straight line La and the straight line Lb of the inner layer protrusion part 28a is smaller than the angle θ2 formed between the straight line Lc and the straight line Ld of the outer layer protrusion part 28b, the inclination of the inner layer protrusion part 28a at the base ends 28B side becomes gentler than the inclination of the outer layer protrusion part 28b. Therefore, when the balloon 2 is inflated by being pressurized, the inner layer protrusion part 28a, which is composed of a material having a lower Shore D hardness than that of the outer layer protrusion part 28b, is more likely to stretch in the circumferential direction z1. In the protrusion part 28, since the inner layer protrusion part 28a stretches preferentially over the outer layer protrusion part 28b, elongation in the circumferential direction z1 of the outer layer protrusion part 28b is suppressed. As a result, deformation of the outer layer protrusion part 28b is prevented, making deformation of the outer shape of the protrusion part 28 less likely to occur, thereby enabling safe and efficient incision of the stenosis while improving the safety of treatment or procedures with the balloon catheter 1.

In a cross section perpendicular to the longitudinal axis direction x1 at the straight tubular part 23, the angle θ1 formed in the first direction d1 in the circumferential direction z1 between the straight line La that connects the two inner layer ends 28aB and the straight line Lb that connects the inner layer apex 28aT and one of the two inner layer ends 28aB may be 0.98 times or less the angle θ2 formed in the first direction d1 in the circumferential direction z1 between the straight line Lc that connects the two outer layer ends 28bB and the straight line Ld that connects the outer layer apex 28bT and one of the two outer layer ends 28bB, 0.95 times or less, 0.90 times or less, or 0.85 times or less. By setting the upper limit of the ratio of the angle θ1 formed between the straight lines La and Lb to the angle θ2 formed between the straight lines Lc and Ld within the above range, the effect of making the inner layer protrusion part 28a stretch more easily than the outer layer protrusion part 28b in the circumferential direction z1 can be enhanced. In addition, in the cross section perpendicular to the longitudinal axis direction x1 at the straight tubular part 23, the angle θ1 formed in the first direction d1 in the circumferential direction z1 between the straight line La that connects the two inner layer ends 28aB and the straight line Lb that connects the inner layer apex 28aT and one of the two inner layer ends 28aB may be 0.10 times or more the angle θ2 formed in the first direction d1 in the circumferential direction z1 between the straight line Lc that connects the two outer layer ends 28bB and the straight line Ld that connects the outer layer apex 28bT and one of the two outer layer ends 28bB, 0.15 times or more, or 0.20 times or more. By setting the lower limit of the ratio of the angle θ1 formed between the straight lines La and Lb to the angle θ2 formed between the straight lines Lc and Ld within the above range, the thickness of the inner layer 20a at the inner layer apex 28aT can be increased, thereby making it more difficult for the inner layer protrusion part 28a to rupture when the balloon 2 is inflated and the inner layer protrusion part 28a is stretched in the circumferential direction z1.

In a cross section perpendicular to the longitudinal axis direction x1 at the straight tubular part 23, the angle θ1 formed in the first direction d1 in the circumferential direction z1 between the straight line La that connects the two inner layer ends 28aB and the straight line Lb that connects the inner layer apex 28aT and one of the two inner layer ends 28aB may be 5 degrees or more, 10 degrees or more, or 15 degrees or more. By setting the lower limit of the angle θ1 in the straight tubular part 23 within the above range, it is possible to ensure the thickness of the inner layer 20a at the inner layer apex 28aT in the protrusion part 28 of the straight tubular part 23, thereby making it more difficult for the inner layer protrusion part 28a to rupture when the balloon 2 is inflated and the inner layer protrusion part 28a is stretched in the circumferential direction z1. In addition, in the cross section perpendicular to the longitudinal axis direction x1 at the straight tubular part 23, the angle θ1 formed in the first direction d1 in the circumferential direction z1 between the straight line La that connects the two inner layer ends 28aB and the straight line Lb that connects the inner layer apex 28aT and one of the two inner layer ends 28aB may be 60 degrees or less, 50 degrees or less, or 40 degrees or less. By setting the upper limit of the angle θ1 in the straight tubular part 23 within the above range, it becomes easier to make the thickness of the outer layer 20b at the outer layer apex 28bT greater than the thickness of the inner layer 20a at the inner layer apex 28aT in the protrusion part 28 of the straight tubular part 23, thereby enhancing the rigidity of the protrusion part 28 and allowing it to more easily bite into the stenotic site.

In a cross section perpendicular to the longitudinal axis direction x1 at the straight tubular part 23, the angle θ2 formed in the first direction d1 in the circumferential direction z1 between the straight line Lc that connects the two outer layer ends 28bB and the straight line Ld that connects the outer layer apex 28bT and one of the two outer layer ends 28bB may be 30 degrees or more, 35 degrees or more, or 40 degrees or more. By setting the lower limit of the angle θ2 in the straight tubular part 23 within the above range, it becomes easier to increase the thickness of the outer layer 20b at the outer layer apex 28bT in the protrusion part 28 of the straight tubular part 23, thereby enhancing the rigidity of the protrusion part 28 and making it possible to form a protrusion part 28 that facilitates the incision of the stenotic site. In addition, in the cross section perpendicular to the longitudinal axis direction x1 at the straight tubular part 23, the angle θ2 formed in the first direction d1 in the circumferential direction z1 between the straight line Lc that connects the two outer layer ends 28bB and the straight line Ld that connects the outer layer apex 28bT and one of the two outer layer ends 28bB may be 80 degrees or less, 75 degrees or less, or 70 degrees or less. By setting the upper limit of the angle θ2 in the straight tubular part 23 within the above range, it becomes possible to sharpen the shape of the apex of the protrusion part 28 while maintaining the rigidity of the protrusion part 28, thereby allowing the protrusion part 28 to more easily pierce into the stenotic site.

As shown in FIG. 2, the balloon 2 may have a two-layer structure composed of at least the inner layer 20a and the outer layer 20b throughout the straight tubular part 23. That is, in the straight tubular part 23, at least the inner layer 20a and the outer layer 20b may be continuously present over the entire 360 degrees in the circumferential direction z1 from a portion where the protrusion part 28 is not provided to a portion where the protrusion part 28 is provided. Because the balloon 2 has a two-layer structure composed of at least the inner layer 20a and the outer layer 20b throughout the straight tubular part 23, it is possible to improve the scoring function of the protrusion part 28, the strength of the balloon 2, and the deliverability, all by means of the outer layer 20b having high Shore D hardness.

The balloon 2 may further include a layer different from the inner layer 20a and the outer layer 20b. As specific examples, although not shown in the figures, the balloon 2 may include an innermost layer located inward in the radial direction y1 from the inner layer 20a, an outermost layer located outward in the radial direction y1 from the outer layer 20b, or an intermediate layer located outward in the radial direction y1 from the inner layer 20a and inward in the radial direction y1 from the outer layer 20b.

The protrusion part 28 and the balloon body part 20 may be integrally formed. The integrally formed protrusion part 28 and balloon body part 20 can prevent the protrusion part 28 from detaching from the balloon body part 20.

Even when an inner protrusion part is provided, the inner layer 20a and the outer layer 20b in a portion of the balloon 2 where the inner protrusion part is not provided and in a portion where the inner protrusion part is provided may be continuous in the circumferential direction z1. This continuity allows the inner protrusion part and the balloon body part 20 to be integrally formed, thereby preventing the inner protrusion part from detaching from the balloon body part 20.

As shown in FIG. 2, in a cross section perpendicular to the longitudinal axis direction x1 at the straight tubular part 23, the inner layer apex 28aT may be located outward in the radial direction y1 relative to the straight line Lc connecting the two outer layer ends 28bB. The inner layer apex 28aT, which is located outward in the radial direction y1 relative to the straight line Lc connecting the two outer layer ends 28bB at the straight tubular part 23, allows the inner layer 20a to have an increased thickness at the inner layer apex 28aT in the protrusion part 28. As a result, when the balloon 2 is inflated, the inner layer protrusion part 28a becomes more stretchable in the circumferential direction z1 than the outer layer protrusion part 28b, and the outer shape of the protrusion part 28 becomes less likely to deform.

As shown in FIG. 2, in a cross section perpendicular to the longitudinal axis direction x1 at the straight tubular part 23, an angle θa at the inner layer apex 28aT in a triangle formed by connecting the two inner layer ends 28aB and the inner layer apex 28aT may be an obtuse angle, and an angle θb at the outer layer apex 28bT in a triangle formed by connecting the two outer layer ends 28bB and the outer layer apex 28bT may be an acute angle. That is, in the protrusion part 28 in the cross section perpendicular to the longitudinal axis direction x1 at the straight tubular part 23, the angle θa, which is the interior angle at the apex of the triangle formed by the two inner layer ends 28aB and the inner layer apex 28aT, may be greater than 90 degrees and less than 180 degrees, and the angle θb, which is the interior angle at the apex of the triangle formed by the two outer layer ends 28bB and the outer layer apex 28bT, may be greater than 0 degrees and less than 90 degrees. When the angle θa at the inner layer apex 28aT in the triangle formed by the two inner layer ends 28aB and the inner layer apex 28aT is an obtuse angle, the entire inner layer protrusion part 28a easily elongates in the circumferential direction z1 when the balloon 2 is inflated under pressure, thereby enhancing the effect of preventing deformation of the outer shape of the protrusion part 28. In addition, when the angle θb at the outer layer apex 28bT in the triangle formed by the two outer layer ends 28bB and the outer layer apex 28bT is an acute angle, the apex of the protrusion part 28 takes a sharp shape, making it easier for the protrusion part 28 to pierce into the stenosis. Thus, when the angle θa at the inner layer apex 28aT is an obtuse angle and the angle θb at the outer layer apex 28bT is an acute angle, it becomes possible to provide the balloon 2 that exhibits a protrusion part 28 which is resistant to deformation during balloon inflation and easily penetrates the stenosis, thereby enabling efficient incision of the stenosis.

In a cross section perpendicular to the longitudinal axis direction x1 at the straight tubular part 23, the angle θa at the inner layer apex 28aT in a triangle formed by connecting the two inner layer ends 28aB and the inner layer apex 28aT may be 90 degrees or more, 100 degrees or more, 110 degrees or more, or 120 degrees or more. By setting the lower limit value of the angle θa within the above range, the inclination between the inner layer ends 28aB and the inner layer apex 28aT becomes gentle, so that the entire inner layer 20a in the inner layer protrusion part 28a is more likely to elongate in the circumferential direction z1, thereby making it easier to prevent deformation of the outer shape of the protrusion part 28. The thickness of the inner layer protrusion part 28a becomes larger, and the inner layer protrusion part 28a is more likely to elongate in the circumferential direction z1 than the outer layer protrusion part 28b. In a cross section perpendicular to the longitudinal axis direction x1 at the straight tubular part 23, the angle θa at the inner layer apex 28aT in the triangle formed by connecting the two inner layer ends 28aB and the inner layer apex 28aT may be 170 degrees or less, 160 degrees or less, 150 degrees or less, or 130 degrees or less. By setting the upper limit value of the angle θa within the above range, the thickness of the inner layer 20a at the inner layer apex 28aT becomes larger, and the inner layer protrusion part 28a becomes more likely to elongate in the circumferential direction z1 than the outer layer protrusion part 28b.

In a cross section perpendicular to the longitudinal axis direction x1 at the straight tubular part 23, the angle θb at the outer layer apex 28bT in a triangle formed by connecting the two outer layer ends 28bB and the outer layer apex 28bT may be 10 degrees or more, 20 degrees or more, or 30 degrees or more. By setting the lower limit value of the angle θb within the above range, it becomes possible to prevent the outer shape of the outer layer apex 28bT from becoming excessively sharp, and the strength of the outer layer apex 28bT can be improved. As a result, even if other objects such as the inner wall of a blood vessel come into contact with the protrusion part 28, the apex of the protrusion part 28 is less likely to deform, which enables improved efficiency of incision into the stenosis. In a cross section perpendicular to the longitudinal axis direction x1 at the straight tubular part 23, the angle θb at the outer layer apex 28bT in a triangle formed by connecting the two outer layer ends 28bB and the outer layer apex 28bT may be 85 degrees or less, 80 degrees or less, or 75 degrees or less. By setting the upper limit value of the angle θb within the above range, the outer layer apex 28bT can be made sharp, thereby allowing the protrusion part 28 to more easily pierce into the stenosis and enhance the efficiency of incision.

As shown in FIG. 2, in a cross section perpendicular to the longitudinal axis direction x1 at the straight tubular part 23, the area of the inner layer protrusion part 28a may be smaller than the area of the outer layer protrusion part 28b. The smaller area of the inner layer protrusion part 28a compared to that of the outer layer protrusion part 28b results in a greater proportion of the outer layer 20b than the inner layer 20a in the protrusion part 28. This greater proportion of the outer layer 20b, which has a higher Shore D hardness than the inner layer 20a, increases the rigidity of the protrusion part 28, making it more likely to bite into the stenosis and enabling the stenosis to be incised more efficiently.

In a cross section perpendicular to the longitudinal axis direction x1 at the straight tubular part 23, the area of the inner layer protrusion part 28a may be 90% or less of the area of the outer layer protrusion part 28b, 80% or less, or 70% or less. By setting the upper limit of the ratio between the area of the inner layer protrusion part 28a and the area of the outer layer protrusion part 28b within the above range, the proportion of the outer layer 20b relative to the inner layer 20a in the protrusion part 28 can be increased, thereby improving the rigidity of the protrusion part 28. In addition, in the cross section perpendicular to the longitudinal axis direction x1 at the straight tubular part 23, the area of the inner layer protrusion part 28a may be 5% or more of the area of the outer layer protrusion part 28b, 10% or more, or 15% or more. By setting the lower limit of the ratio between the area of the inner layer protrusion part 28a and the area of the outer layer protrusion part 28b within the above range, the area of the inner layer 20a in the protrusion part 28 can be secured, and the inner layer protrusion part 28a can be made more likely to extend in the circumferential direction z1 than the outer layer protrusion part 28b during expansion of the balloon 2, thereby enhancing the effect of preventing deformation of the outer shape of the protrusion part 28.

In a cross section perpendicular to the longitudinal axis direction x1 at at least one of the proximal tapered part 22 and the distal tapered part 24, the protrusion part 28 provided on at least one of the proximal tapered part 22 and the distal tapered part 24 may be configured to be tilted in either the first direction d1 or the second direction d2 of the circumferential direction z1, or may be configured not to be tilted in either direction. When the protrusion part 28 is not tilted in either the first direction d1 or the second direction d2 of the circumferential direction z1 at at least one of the proximal tapered part 22 and the distal tapered part 24, the protrusion part 28 increases the rigidity of the balloon 2 in the longitudinal axis direction x1 at the proximal tapered part 22 or the distal tapered part 24 where the protrusion part 28 is provided. As a result, the insertability of the balloon 2 into a blood vessel lumen can be improved. On the other hand, when the protrusion part 28 is tilted in either the first direction d1 or the second direction d2 of the circumferential direction z1 at at least one of the proximal tapered part 22 and the distal tapered part 24, the apex 28T of the protrusion part 28 at the proximal tapered part 22 or the distal tapered part 24 is less likely to come into contact with another object such as the wall of the blood vessel lumen during delivery of the balloon 2 to the lesion, thereby preventing damage to the vessel wall.

Although not shown in the figures, in a cross section perpendicular to the longitudinal axis direction x1 at at least one of the proximal tapered part 22 and the distal tapered part 24, a distal end portion of the protrusion part 28, which is located outward in the radial direction y1, and which is provided at at least one of the proximal tapered part 22 and the distal tapered part 24, may be removed by processing such as cutting, dissolving, or crushing. The presence of a protrusion part 28, in which the distal end portion located outward in the radial direction y1 is removed, at at least one of the proximal tapered part 22 and the distal tapered part 24, makes it possible to reduce the risk of damaging the inner wall of a blood vessel even when the protrusion part 28 of the proximal tapered part 22 or the distal tapered part 24 comes into contact with the inner wall of the blood vessel while the balloon 2 is being inserted into the lumen of the blood vessel. As a result, it is possible to provide the balloon 2 with improved safety.

When the distal end portion on the radially outward side of the protrusion part 28 provided on at least one of the proximal tapered part 22 and the distal tapered part 24 is removed, the protrusion part 28 in at least one of the proximal tapered part 22 and the distal tapered part 24, in which the distal end portion of the protrusion part 28 is removed, may include an outer layer protrusion part 28b and an inner layer protrusion part 28a. In other words, in the protrusion part 28 provided on at least one of the proximal tapered part 22 and the distal tapered part 24, the outer layer apex 28bT may be removed while a portion of the outer layer protrusion part 28b including the outer layer ends 28bB remains. The presence of the outer layer protrusion part 28b and the inner layer protrusion part 28a in the protrusion part 28, where the distal end portion of the protrusion part 28 is removed, makes it possible to prevent damage to the inner wall of a blood vessel even when the protrusion part 28 of the proximal tapered part 22 or the distal tapered part 24 comes into contact with the inner wall. This also allows the outer layer 20b to increase the stiffness in the longitudinal axis direction x1 of the proximal tapered part 22 or the distal tapered part 24, thereby improving the insertability of the balloon 2 into the vascular lumen.

As shown in FIG. 3, in a cross section perpendicular to the longitudinal axis direction x1 at at least one of the proximal tapered part 22 and the distal tapered part 24, the angle θ3 formed between the straight line La connecting the two inner layer ends 28aB and the straight line Lb connecting one of the inner layer ends 28aB and the inner layer apex 28aT in the first direction d1 of the circumferential direction z1 may be smaller than the angle θ4 formed between the straight line Lc connecting the two outer layer ends 28bB and the straight line Ld connecting one of the outer layer ends 28bB and the outer layer apex 28bT in the first direction d1 of the circumferential direction z1. When the angle θ3 formed by the straight lines La and Lb of the inner layer protrusion part 28a is smaller than the angle θ4 formed by the straight lines Lc and Ld of the outer layer protrusion part 28b at at least one of the proximal tapered part 22 and the distal tapered part 24, the inclination of the inner layer protrusion part 28a on the base ends 28B side becomes gentler than the inclination of the outer layer protrusion part 28b. As a result, when the balloon 2 is inflated, the inner layer protrusion part 28a becomes more likely to stretch in the circumferential direction z1 than the outer layer protrusion part 28b even at the proximal tapered part 22 or the distal tapered part 24, thereby making it possible to prevent the outer shape of the protrusion part 28 from being deformed from the proximal tapered part 22 or the distal tapered part 24 to the straight tubular part 23.

In a cross section perpendicular to the longitudinal axis direction x1 at at least one of the proximal tapered part 22 and the distal tapered part 24, the angle θ3 formed by the straight line La connecting the two inner layer ends 28aB and the straight line Lb connecting the inner layer ends 28aB and the inner layer apex 28aT in the first direction d1 of the circumferential direction z1 may be 0.95 times or less the angle θ4 formed by the straight line Lc connecting the two outer layer ends 28bB and the straight line Ld connecting the outer layer ends 28bB and the outer layer apex 28bT in the first direction d1 of the circumferential direction z1, 0.90 times or less the angle θ4, or 0.85 times or less the angle θ4. By setting the upper limit value of the ratio of the angle θ3 formed by the straight lines La and Lb to the angle θ4 formed by the straight lines Lc and Ld within the above range, it becomes possible to make the inner layer protrusion part 28a more likely to stretch in the circumferential direction z1 than the outer layer protrusion part 28b. Also, in a cross section perpendicular to the longitudinal axis direction x1 at at least one of the proximal tapered part 22 and the distal tapered part 24, the angle θ3 formed by the straight line La connecting the two inner layer ends 28aB and the straight line Lb connecting the inner layer ends 28aB and the inner layer apex 28aT in the first direction d1 of the circumferential direction z1 may be 0.10 times or more the angle θ4 formed by the straight line Lc connecting the two outer layer ends 28bB and the straight line Ld connecting the outer layer ends 28bB and the outer layer apex 28bT in the first direction d1 of the circumferential direction z1, 0.15 times or more the angle θ4, or 0.20 times or more the angle θ4. By setting the lower limit value of the ratio of the angle θ3 formed by the straight lines La and Lb to the angle θ4 formed by the straight lines Lc and Ld within the above range, it becomes possible to increase the thickness of the inner layer 20a at the inner layer apex 28aT, and to make the inner layer protrusion part 28a less likely to rupture when the balloon 2 is expanded and the inner layer protrusion part 28a is stretched in the circumferential direction z1.

In a cross section perpendicular to the longitudinal axis direction x1 at at least one of the proximal tapered part 22 and the distal tapered part 24, the angle θ3 formed by the straight line La connecting the two inner layer ends 28aB and the straight line Lb connecting the inner layer ends 28aB and the inner layer apex 28aT in the first direction d1 of the circumferential direction z1 may be 10 degrees or more, 15 degrees or more, 20 degrees or more, or 25 degrees or more. By setting the lower limit value of the angle θ3 at at least one of the proximal tapered part 22 and the distal tapered part 24 within the above range, it becomes possible to increase the thickness of the inner layer 20a at the inner layer apex 28aT in the proximal tapered part 22 or the distal tapered part 24, and to enhance the flexibility of the protrusion part 28 so that it is less likely to damage the vessel lumen wall even when it comes into contact with it. In a cross section perpendicular to the longitudinal axis direction x1 at at least one of the proximal tapered part 22 and the distal tapered part 24, the angle θ3 formed by the straight line La connecting the two inner layer ends 28aB and the straight line Lb connecting the inner layer ends 28aB and the inner layer apex 28aT in the first direction d1 of the circumferential direction z1 may be 65 degrees or less, 60 degrees or less, or 55 degrees or less. By setting the upper limit value of the angle θ3 at at least one of the proximal tapered part 22 and the distal tapered part 24 within the above range, it becomes possible to prevent the height of the protrusion part 28 from becoming excessively large in the proximal tapered part 22 or the distal tapered part 24, and to make it less likely for the protrusion part 28 to come into contact with the vessel lumen wall.

In a cross section perpendicular to the longitudinal axis direction x1 at at least one of the proximal tapered part 22 and the distal tapered part 24, the angle θ4 formed by the straight line Lc connecting the two outer layer ends 28bB and the straight line Ld connecting the outer layer ends 28bB and the outer layer apex 28bT in the first direction d1 of the circumferential direction z1 may be 30 degrees or more, 35 degrees or more, or 40 degrees or more. By setting the lower limit of the angle θ4 at at least one of the proximal tapered part 22 and the distal tapered part 24 within the above range, it becomes possible to ensure the thickness of the outer layer 20b at the protrusion part 28 in the proximal tapered part 22 or the distal tapered part 24, enhance the rigidity in the longitudinal axis direction x1, and thereby improve the insertability of the balloon 2 into the vessel lumen. In addition, the angle θ4 formed by the straight line Lc connecting the two outer layer ends 28bB and the straight line Ld connecting the outer layer ends 28bB and the outer layer apex 28bT in the first direction d1 of the circumferential direction z1 may be 85 degrees or less, 80 degrees or less, or 75 degrees or less. By setting the upper limit of the angle θ4 at at least one of the proximal tapered part 22 and the distal tapered part 24 within the above range, it becomes easier to increase the thickness of the inner layer 20a at the protrusion part 28 in the proximal tapered part 22 or the distal tapered part 24, making it possible to provide a protrusion part 28 that is highly flexible and less likely to damage the vessel lumen wall upon contact.

In a cross section perpendicular to the longitudinal direction x1 at at least one of the proximal sleeve part 21 and the distal sleeve part 25, the protrusion part 28 provided in at least one of the proximal sleeve part 21 and the distal sleeve part 25 may be configured to be not tilted in either of the first direction d1 and the second direction d2 of the circumferential direction z1, or may be configured to be tilted in either of the first direction d1 or the second direction d2 of the circumferential direction z1. By having the protrusion part 28 not tilted in either of the first direction d1 and the second direction d2 of the circumferential direction z1 at at least one of the proximal sleeve part 21 and the distal sleeve part 25, the rigidity of the balloon 2 in the longitudinal direction x1 is increased by the protrusion part 28 at the proximal sleeve part 21 or the distal sleeve part 25 where the protrusion part 28 is provided, and the insertability of the balloon 2 into a blood vessel lumen can be improved. By having the protrusion part 28 tilted in either of the first direction d1 or the second direction d2 of the circumferential direction z1 at at least one of the proximal sleeve part 21 and the distal sleeve part 25, the apex 28T of the protrusion part 28 of the proximal sleeve part 21 or the distal sleeve part 25 is less likely to contact the inner wall of the blood vessel when the balloon 2 is delivered to a lesion. Therefore, the protrusion part 28 of the proximal sleeve part 21 or the distal sleeve part 25 can prevent the inner wall of the blood vessel from being damaged.

Although not shown, in a cross section perpendicular to the longitudinal direction x1 at at least one of the proximal sleeve part 21 and the distal sleeve part 25, the protrusion part 28 provided in at least one of the proximal sleeve part 21 and the distal sleeve part 25 may have its distal end portion on the outer side in the radial direction y1 removed by processing such as cutting, dissolving, or crushing. The protrusion part 28 provided in at least one of the proximal sleeve part 21 and the distal sleeve part 25, with the distal end portion removed, makes it possible to reduce the likelihood of damaging the inner wall of the blood vessel or other tissues, even when the protrusion part 28 of the proximal sleeve part 21 or the distal sleeve part 25 comes into contact with such tissues while the balloon 2 is being inserted into a blood vessel. This allows the balloon 2 to be configured with high safety, making it less likely to damage the inner wall of the blood vessel or the like.

In a case where the distal end portion on the outer side in the radial direction y1 is removed in the protrusion part 28 provided in at least one of the proximal sleeve part 21 and the distal sleeve part 25, the protrusion part 28 in at least one of the proximal sleeve part 21 and the distal sleeve part 25, in which the distal end portion of the protrusion part 28 is removed, may include the outer layer protrusion part 28b and the inner layer protrusion part 28a. That is, in the protrusion part 28 provided on at least one of the proximal sleeve part 21 and the distal sleeve part 25, the outer layer apex 28bT may be removed, and a part of the outer layer protrusion part 28b including the outer layer ends 28bB may remain. By having both the outer layer protrusion part 28b and the inner layer protrusion part 28a in at least one of the proximal sleeve part 21 and the distal sleeve part 25 where the distal end portion of the protrusion part 28 is removed, it becomes possible to prevent the inner wall of the blood vessel lumen from being damaged even if the protrusion part 28 of the proximal sleeve part 21 or the distal sleeve part 25 comes into contact with the vessel wall when the balloon 2 passes through the vessel lumen, thereby providing a balloon 2 with high safety. In addition, the longitudinal rigidity of the proximal sleeve part 21 and the distal sleeve part 25 can be enhanced by the outer layer 20b, making it possible to improve the insertability of the balloon 2 into the vessel lumen.

In a cross section perpendicular to the longitudinal direction x1 at at least one of the proximal sleeve part 21 and the distal sleeve part 25, the angle θ5 formed between the straight line La connecting the two inner layer ends 28aB and the straight line Lb connecting the inner layer ends 28aB and the inner layer apex 28aT in the first direction d1 of the circumferential direction z1 may be greater than the angle θ6 formed between the straight line Lc connecting the two outer layer ends 28bB and the straight line Ld connecting the outer layer ends 28bB and the outer layer apex 28bT in the first direction d1 of the circumferential direction z1. Because the angle θ5 formed between the straight lines La and Lb of the inner layer protrusion part 28a is greater than the angle θ6 formed between the straight lines Lc and Ld of the outer layer protrusion part 28b at at least one of the proximal sleeve part 21 and the distal sleeve part 25, the thickness of the inner layer 20a at the inner layer apex 28aT becomes greater in the protrusion part 28. Since the Shore D hardness of the inner layer 20a is lower than that of the outer layer 20b, the elasticity of the protrusion part 28 can be enhanced. As a result, when the balloon 2 is inserted into a blood vessel lumen, even if the protrusion part 28 of the proximal sleeve part 21 or the distal sleeve part 25 comes into contact with the inner wall of the blood vessel lumen, it becomes less likely to damage the wall by the protrusion part 28.

In a cross section perpendicular to the longitudinal direction x1 at at least one of the proximal sleeve part 21 and the distal sleeve part 25, the angle θ5 formed between the straight line La connecting the two inner layer ends 28aB and the straight line Lb connecting the inner layer ends 28aB and the inner layer apex 28aT in the first direction d1 of the circumferential direction z1 may be 1.05 times or more the angle θ6 formed between the straight line Lc connecting the two outer layer ends 28bB and the straight line Ld connecting the outer layer ends 28bB and the outer layer apex 28bT in the first direction d1 of the circumferential direction z1, 1.10 times or more, or 1.15 times or more. By setting the lower limit of the ratio of the angle θ5 to the angle θ6 within the above range, the thickness of the inner layer 20a at the inner layer apex 28aT can be increased, thereby enhancing the elasticity of the protrusion part 28 and improving its ability to avoid damaging the inner wall of a blood vessel lumen. The angle θ5 may be 5 times or less the angle θ6, 4 times or less, or 3 times or less. By setting the upper limit of the ratio of the angle θ5 to the angle θ6 within the above range, the thickness of the outer layer apex 28bT in the protrusion part 28 can be maintained to some extent, and the longitudinal rigidity of the balloon 2 at the proximal sleeve part 21 or the distal sleeve part 25 can be maintained, thereby improving insertability.

In a cross section perpendicular to the longitudinal axis direction x1 at at least one of the proximal sleeve part 21 and the distal sleeve part 25, the angle θ5 formed in the first direction d1 in the circumferential direction z1 between the straight line La connecting the two inner layer ends 28aB and the straight line Lb connecting the inner layer apex 28aT and one of the two inner layer ends 28aB may be 10 degrees or more, 20 degrees or more, 25 degrees or more, 30 degrees or more, or 35 degrees or more. By setting the lower limit of the angle θ5 to the above range at at least one of the proximal sleeve part 21 and the distal sleeve part 25, it becomes possible to increase the thickness of the inner layer 20a at the inner layer apex 28aT in the proximal sleeve part 21 and/or the distal sleeve part 25, and to provide a protrusion part 28 that is flexible and less likely to damage the vessel wall when in contact. Also, in a cross section perpendicular to the longitudinal axis direction x1 at at least one of the proximal sleeve part 21 and the distal sleeve part 25, the angle θ5 formed in the first direction d1 in the circumferential direction z1 between the straight line La connecting the two inner layer ends 28aB and the straight line Lb connecting the inner layer apex 28aT and one of the two inner layer ends 28aB may be 80 degrees or less, 75 degrees or less, 70 degrees or less, or 60 degrees or less. By setting the upper limit of the angle θ5 to the above range at at least one of the proximal sleeve part 21 and the distal sleeve part 25, it is possible to prevent the height of the protrusion part 28 from becoming excessively high and to make it less likely for the protrusion part 28 to come into contact with the inner wall of the blood vessel.

In a cross section perpendicular to the longitudinal axis direction x1 in at least one of the proximal sleeve part 21 and the distal sleeve part 25, the angle θ6 formed between the straight line Lc connecting the two outer layer ends 28bB and the straight line Ld connecting one of the two outer layer ends 28bB and the outer layer apex 28bT in the first direction d1 of the circumferential direction z1 may be 10 degrees or more, 20 degrees or more, 30 degrees or more, 35 degrees or more, or 40 degrees or more. By setting the lower limit value of the angle θ6 in at least one of the proximal sleeve part 21 and the distal sleeve part 25 within the above range, it becomes possible to ensure the thickness of the outer layer 20b at the protrusion part 28 in the proximal sleeve part 21 or the distal sleeve part 25. This allows the rigidity in the longitudinal axis direction x1 in the proximal sleeve part 21 or the distal sleeve part 25 to be increased, thereby improving the insertability of the balloon 2 into a blood vessel lumen. In a cross section perpendicular to the longitudinal axis direction x1 in at least one of the proximal sleeve part 21 and the distal sleeve part 25, the angle θ6 formed between the straight line Lc connecting the two outer layer ends 28bB and the straight line Ld connecting one of the two outer layer ends 28bB and the outer layer apex 28bT in the first direction d1 of the circumferential direction z1 may be 90 degrees or less, 85 degrees or less, or 80 degrees or less. By setting the upper limit value of the angle θ6 in at least one of the proximal sleeve part 21 and the distal sleeve part 25 within the above range, the thickness of the inner layer 20a at the protrusion part 28 in the proximal sleeve part 21 or the distal sleeve part 25 can be increased, and the flexibility of the protrusion part 28 can be enhanced, so that the protrusion part 28 is less likely to damage the blood vessel lumen wall even when it comes into contact therewith.

As shown in FIG. 4, in a cross section perpendicular to the longitudinal axis direction x1 in at least one of the proximal sleeve part 21 and the distal sleeve part 25, the angle θ5 formed between the straight line La connecting the two inner layer ends 28aB and the straight line Lb connecting one of the two inner layer ends 28aB and the inner layer apex 28aT in the first direction d1 of the circumferential direction z1 may be smaller than the angle θ6 formed between the straight line Lc connecting the two outer layer ends 28bB and the straight line Ld connecting one of the two outer layer ends 28bB and the outer layer apex 28bT in the first direction d1 of the circumferential direction z1. When the angle θ5 formed between the straight line La and the straight line Lb of the inner layer protrusion part 28a is smaller than the angle θ6 formed between the straight line Lc and the straight line Ld of the outer layer protrusion part 28b in at least one of the proximal sleeve part 21 and the distal sleeve part 25, the inclination of the inner layer protrusion part 28a on the base end 28B side becomes gentler than the inclination of the outer layer protrusion part 28b, making the inner layer protrusion part 28a more likely to elongate in the circumferential direction z1 than the outer layer protrusion part 28b in the proximal sleeve part 21 or the distal sleeve part 25. As a result, when the balloon 2 is pressurized and expanded, it becomes possible to prevent the shape of the protrusion part 28 in the proximal sleeve part 21 or the distal sleeve part 25 from being deformed.

Next, a second balloon for a balloon catheter will be described. In the following explanation of the second balloon for a balloon catheter, descriptions that overlap with those of the first balloon for a balloon catheter described above are omitted.

A second balloon for a balloon catheter according to one or more embodiments of the present invention has a longitudinal axis direction, a radial direction, and a circumferential direction, and has an outer layer and an inner layer composed of a material having a Shore D hardness lower than that of the outer layer; the balloon has a straight tubular part, a proximal tapered part located proximal to the straight tubular part, a proximal sleeve part located proximal to the proximal tapered part, a distal tapered part located distal to the straight tubular part, and a distal sleeve part located distal to the distal tapered part; the balloon further has a protrusion part that protrudes outward in the radial direction and extends in the longitudinal axis direction, wherein, in a cross section perpendicular to the longitudinal axis direction at the straight tubular part, a region where the protrusion is present includes an outer layer protrusion part formed by the outer layer and protruding outward in the radial direction and an inner layer protrusion part formed by the inner layer and protruding outward in the radial direction; the outer layer protrusion part includes an outer layer apex being an apex of the outer layer protrusion part and outer layer ends located on both sides in the circumferential direction at respective circumferential ends of the outer layer protrusion part; the inner layer protrusion part includes an inner layer apex being an apex of the inner layer protrusion part and inner layer ends located on both sides in the circumferential direction at respective circumferential ends of the inner layer protrusion part; and a ratio (θ2/θ1) of an angle θ2 formed in the first direction in the circumferential direction between a straight line that connects the two outer layer ends and a straight line that connects the outer layer apex and one of the two outer layer ends to an angle θ1 formed in the first direction in the circumferential direction between a straight line that connects the two inner layer ends and a straight line that connects the inner layer apex and one of the two inner layer ends, in the cross section perpendicular to the longitudinal axis direction at the straight tubular part, is greater than a ratio (θ4/θ3) of an angle θ4 formed in the first direction in the circumferential direction between a straight line that connects the two outer layer ends and a straight line that connects the outer layer apex and one of the two outer layer ends to an angle θ3 formed in the first direction in the circumferential direction between a straight line that connects the two inner layer ends and a straight line that connects the inner layer apex and one of the two inner layer ends, in a cross section perpendicular to the longitudinal axis direction at at least one of the proximal tapered part and the distal tapered part.

When the balloon moves forward or backward in the blood vessel lumen, there is a risk that the blood vessel wall may be damaged due to the protrusion parts provided at both longitudinal ends of the balloon, such as the proximal tapered part and the distal tapered part, coming into contact with the blood vessel lumen. However, according to the above balloon for a balloon catheter, which includes an outer layer and an inner layer made of a material having a Shore D hardness lower than that of the outer layer, since the ratio of angle θ2 of the outer layer protrusion part to angle θ1 of the inner layer protrusion part in the straight tubular part is greater than the ratio of angle θ4 of the outer layer protrusion part to angle θ3 of the inner layer protrusion part at at least one of the proximal tapered part and the distal tapered part, it is possible to enhance the cushioning property of the protrusion part at the proximal tapered part and the distal tapered part. As a result, even if the protrusion part of the proximal tapered part or the distal tapered part comes into contact with the blood vessel wall, it is less likely to cause damage, and it is possible to prevent damage to the blood vessel wall. Furthermore, since the rigidity of the protrusion part in the straight tubular part can be increased compared to that of the protrusion part in the proximal tapered part or the distal tapered part, it is possible to improve the deliverability through the blood vessel lumen and the incision efficiency of the stenosed region. This makes it possible to perform efficient incision of the stenosed region while improving the safety of treatment or procedures using the balloon catheter.

As shown in FIGS. 2 and 3, in a cross section perpendicular to the longitudinal axis x1 at the straight tubular part 23, the ratio (θ2/θ1) of an angle θ2 formed by a straight line that connects the outer layer apex 28bT and one of the two outer layer ends 28bB and a straight line that connects the two outer layer ends 28bB in the first direction d1 of the circumferential direction z1, to an angle θ1 formed by a straight line that connects the inner layer apex 28aT and one of the two inner layer ends 28aB and a straight line that connects the two inner layer ends 28aB in the first direction d1 of the circumferential direction z1, is greater than the ratio (θ4/θ3) of an angle θ4 formed by a straight line that connects the outer layer apex 28bT and one of the two outer layer ends 28bB and a straight line that connects the two outer layer ends 28bB in the first direction d1 of the circumferential direction z1, to an angle θ3 formed by a straight line that connects the inner layer apex 28aT and one of the two inner layer ends 28aB and a straight line that connects the two inner layer ends 28aB in the first direction d1 of the circumferential direction z1, in at least one of the proximal tapered part 22 and the distal tapered part 24.

The ratio of the angle θ2 formed by a straight line that connects the two outer layer ends 28bB and a straight line that connects one of the outer layer ends 28bB and the outer layer apex 28bT in the straight tubular part 23 to the angle θ1 formed by a straight line that connects the two inner layer ends 28aB and a straight line that connects one of the inner layer ends 28aB and the inner layer apex 28aT is greater than the ratio of the angle θ4 formed by a straight line that connects the two outer layer ends 28bB and a straight line that connects one of the outer layer ends 28bB and the outer layer apex 28bT to the angle θ3 formed by a straight line that connects the two inner layer ends 28aB and a straight line that connects one of the inner layer ends 28aB and the inner layer apex 28aT in at least one of the proximal tapered part 22 and the distal tapered part 24. Due to this configuration, the flexibility of the protrusion parts 28 can be increased in the proximal tapered part 22 and the distal tapered part 24, thereby improving the cushioning property of the protrusion parts 28. As a result, even when the protrusion parts 28 of the proximal tapered part 22 and the distal tapered part 24 come into contact with the vascular lumen wall while the balloon 2 moves forward or backward in the vascular lumen, they are less likely to cause damage to the wall, thereby preventing damage to the vascular lumen wall. Furthermore, the rigidity of the protrusion parts 28 in the straight tubular part 23 can be greater than that of the protrusion parts 28 in the proximal tapered part 22 or the distal tapered part 24, making it possible to improve the insertability into the vascular lumen and the efficiency of incising the stenosis. This enables efficient incision of the stenosis while improving the safety of treatment or procedures using the balloon catheter.

In the straight tubular part 23, the ratio of the angle θ2 formed by the straight lines Lc and Ld of the outer layer protrusion part 28b to the angle θ1 formed by the straight lines La and Lb of the inner layer protrusion part 28a may be 1.10 times or more the ratio of the angle θ4 formed by the straight lines Lc and Ld of the outer layer protrusion part 28b to the angle θ3 formed by the straight lines La and Lb of the inner layer protrusion part 28a in at least one of the proximal tapered part 22 and the distal tapered part 24, 1.15 times or more, or 1.20 times or more. By setting the lower limit of the ratio between the ratio of angle θ2 to angle θ1 in the straight tubular part 23 and the ratio of angle θ4 to angle θ3 in at least one of the proximal tapered part 22 and the distal tapered part 24 within the above range, the protrusion part 28 in the proximal tapered part 22 or the distal tapered part 24 can be made more flexible, and the cushioning property of the protrusion part 28 can be enhanced. As a result, the protrusion part 28 of the proximal tapered part 22 or the distal tapered part 24 can be more effective in preventing damage to the inner wall of the blood vessel lumen. Furthermore, the ratio of the angle θ2 formed by the straight lines Lc and Ld of the outer layer protrusion part 28b to the angle θ1 formed by the straight lines La and Lb of the inner layer protrusion part 28a in the straight tubular part 23 may be 3.0 times or less the ratio of the angle θ4 formed by the straight lines Lc and Ld of the outer layer protrusion part 28b to the angle θ3 formed by the straight lines La and Lb of the inner layer protrusion part 28a in at least one of the proximal tapered part 22 and the distal tapered part 24, 2.5 times or less, or 2.0 times or less. By setting the upper limit of the ratio between the ratio of angle θ2 to angle θ1 in the straight tubular part 23 and the ratio of angle θ4 to angle θ3 in at least one of the proximal tapered part 22 and the distal tapered part 24 within the above range, the rigidity of the protrusion part 28 in the straight tubular part 23 can be increased, making it possible to efficiently incise the stenosis by the protrusion part 28 of the straight tubular part 23.

As shown in FIG. 2, in the cross-section perpendicular to the longitudinal axial direction x1 at the straight tubular part 23, the angle θ1 formed in the first direction d1 of the circumferential direction z1 by the straight line La connecting the two inner layer ends 28aB and the straight line Lb connecting the inner layer end 28aB and the inner layer apex 28aT may be smaller than the angle θ2 formed in the first direction d1 of the circumferential direction z1 by the straight line Lc connecting the two outer layer ends 28bB and the straight line Ld connecting the outer layer end 28bB and the outer layer apex 28bT. When the angle θ1 is smaller than the angle θ2 in the straight tubular part 23, the thickness of the outer layer 20b in the protrusion part 28 at the straight tubular part 23 tends to be greater than the thickness of the inner layer 20a, making it possible to increase the rigidity of the protrusion part 28 and enhance its ability to penetrate the stenosis.

As shown in FIG. 3, in the cross-section perpendicular to the longitudinal axial direction x1 at at least one of the proximal taper part 22 and the distal taper part 24, the angle θ3 formed in the first direction d1 of the circumferential direction z1 by the straight line La connecting the two inner layer ends 28aB and the straight line Lb connecting the inner layer end 28aB and the inner layer apex 28aT may be smaller than the angle θ4 formed in the first direction d1 of the circumferential direction z1 by the straight line Lc connecting the two outer layer ends 28bB and the straight line Ld connecting the outer layer end 28bB and the outer layer apex 28bT. When the angle θ3 is smaller than the angle θ4 in at least one of the proximal taper part 22 and the distal taper part 24, the thickness of the outer layer 20b in the proximal taper part 22 and the distal taper part 24 can be more easily ensured, which makes it possible to increase the rigidity in the longitudinal axial direction x1 of the proximal taper part 22 and the distal taper part 24 and thereby improve the insertability of the balloon 2 into the vascular lumen.

As shown in FIGS. 2 and 4, in the cross-section perpendicular to the longitudinal axial direction x1 at the straight tubular part 23, the ratio (θ2/θ1) of the angle θ2 formed in the first direction d1 of the circumferential direction z1 by the straight line Lc connecting the two outer layer ends 28bB and the straight line Ld connecting the outer layer end 28bB and the outer layer apex 28bT to the angle θ1 formed in the first direction d1 of the circumferential direction z1 by the straight line La connecting the two inner layer ends 28aB and the straight line Lb connecting the inner layer end 28aB and the inner layer apex 28aT may be greater than the ratio (06/05) of the angle θ6 formed in the first direction d1 of the circumferential direction z1 by the straight line Lc connecting the two outer layer ends 28bB and the straight line Ld connecting the outer layer end 28bB and the outer layer apex 28bT to the angle θ5 formed in the first direction d1 of the circumferential direction z1 by the straight line La connecting the two inner layer ends 28aB and the straight line Lb connecting the inner layer end 28aB and the inner layer apex 28aT in the cross-section perpendicular to the longitudinal axial direction x1 at at least one of the proximal sleeve part 21 and the distal sleeve part 25.

The ratio of the angle θ2 formed by the straight lines Lc and Ld of the outer layer protrusion part 28b to the angle θ1 formed by the straight lines La and Lb of the inner layer protrusion part 28a at the straight tubular part 23 is greater than the ratio of the angle θ6 formed by the straight lines Lc and Ld of the outer layer protrusion part 28b to the angle θ5 formed by the straight lines La and Lb of the inner layer protrusion part 28a at at least one of the proximal sleeve part 21 and the distal sleeve part 25, whereby the flexibility of the protrusion parts 28 at the proximal sleeve part 21 and the distal sleeve part 25 can be enhanced. When the balloon 2 moves forward or backward in the vascular lumen, the proximal sleeve part 21 and the distal sleeve part 25 become the leading portions. Therefore, although the protrusion parts 28 provided at the proximal sleeve part 21 and the distal sleeve part 25 may contact the vascular lumen and potentially damage the vascular wall, such damage can be prevented due to the flexibility of the protrusion parts 28 at the proximal sleeve part 21 and the distal sleeve part 25.

The ratio of the angle θ2 formed by the straight lines Lc and Ld of the outer layer protrusion part 28b to the angle θ1 formed by the straight lines La and Lb of the inner layer protrusion part 28a in the straight tubular part 23 may be 1.1 times or more, 1.2 times or more, or 1.3 times or more the ratio of the angle θ6 formed by the straight lines Lc and Ld of the outer layer protrusion part 28b to the angle θ5 formed by the straight lines La and Lb of the inner layer protrusion part 28a at at least one of the proximal sleeve part 21 and the distal sleeve part 25. By setting the lower limit of the ratio of the angle θ2 to the angle θ1 in the straight tubular part 23 and the ratio of the angle θ6 to the angle θ5 at at least one of the proximal sleeve part 21 and the distal sleeve part 25 within the above range, the protrusion parts 28 in the proximal sleeve part 21 and the distal sleeve part 25 can be made more flexible than the protrusion parts 28 in the straight tubular part 23. As a result, even when the protrusion parts 28 in the proximal sleeve part 21 and the distal sleeve part 25 come into contact with the vascular lumen wall, the effect of reducing the likelihood of damage can be enhanced. Furthermore, the ratio of the angle θ2 formed by the straight lines Lc and Ld of the outer layer protrusion part 28b to the angle θ1 formed by the straight lines La and Lb of the inner layer protrusion part 28a in the straight tubular part 23 may be 5.0 times or less, 4.5 times or less, or 4.0 times or less the ratio of the angle θ6 formed by the straight lines Lc and Ld of the outer layer protrusion part 28b to the angle θ5 formed by the straight lines La and Lb of the inner layer protrusion part 28a at at least one of the proximal sleeve part 21 and the distal sleeve part 25. By setting the upper limit of the ratio of the angle θ2 to the angle θ1 in the straight tubular part 23 and the ratio of the angle θ6 to the angle θ5 at at least one of the proximal sleeve part 21 and the distal sleeve part 25 within the above range, the rigidity of the protrusion parts 28 in the straight tubular part 23 can be increased, thereby facilitating the incision of the stenosis by the protrusion parts 28 in the straight tubular part 23.

As shown in FIG. 4, the angle θ5 formed in the first direction d1 of the circumferential direction z1 by the straight line La connecting the two inner layer ends 28aB and the straight line Lb connecting the inner layer end 28aB and the inner layer apex 28aT in a cross-section perpendicular to the longitudinal axis direction x1 at at least one of the proximal sleeve part 21 and the distal sleeve part 25 may be smaller than the angle θ6 formed in the first direction d1 of the circumferential direction z1 by the straight line Lc connecting the two outer layer ends 28bB and the straight line Ld connecting the outer layer end 28bB and the outer layer apex 28bT. By making the angle θ5 smaller than the angle θ6 at at least one of the proximal sleeve part 21 and the distal sleeve part 25, it becomes easier to ensure the thickness of the outer layer 20b at the proximal sleeve part 21 and the distal sleeve part 25, thereby increasing the rigidity in the longitudinal axial direction x1 of the proximal sleeve part 21 and the distal sleeve part 25 and improving the insertability of the balloon 2 into the vascular lumen.

As shown in FIGS. 3 and 4, the ratio (θ4/θ3) of the angle θ4 formed by a straight line Lc connecting the two outer layer ends 28bB and a straight line Ld connecting the outer layer end 28bB and the outer layer apex 28bT in the first direction d1 of the circumferential direction z1 in a cross-section perpendicular to the longitudinal direction x1 in at least one of the proximal tapered part 22 and the distal tapered part 24 to the angle θ3 formed by a straight line La connecting the two inner layer ends 28aB and a straight line Lb connecting the inner layer end 28aB and the inner layer apex 28aT in the first direction d1 of the circumferential direction z1 may be greater than the ratio (θ6/θ5) of the angle θ6 formed by a straight line Lc connecting the two outer layer ends 28bB and a straight line Ld connecting the outer layer end 28bB and the outer layer apex 28bT in the first direction d1 of the circumferential direction z1 in a cross-section perpendicular to the longitudinal direction x1 in at least one of the proximal sleeve part 21 and the distal sleeve part 25 to the angle θ5 formed by a straight line La connecting the two inner layer ends 28aB and a straight line Lb connecting the inner layer end 28aB and the inner layer apex 28aT in the first direction d1 of the circumferential direction z1.

When the ratio of the angle θ4 formed by a straight line Lc connecting the two outer layer ends 28bB and a straight line Ld connecting the outer layer end 28bB and the outer layer apex 28bT to the angle θ3 formed by a straight line La connecting the two inner layer ends 28aB and a straight line Lb connecting the inner layer end 28aB and the inner layer apex 28aT in at least one of the proximal tapered part 22 and the distal tapered part 24 is greater than the ratio of the angle θ6 formed by a straight line Lc connecting the two outer layer ends 28bB and a straight line Ld connecting the outer layer end 28bB and the outer layer apex 28bT to the angle θ5 formed by a straight line La connecting the two inner layer ends 28aB and a straight line Lb connecting the inner layer end 28aB and the inner layer apex 28aT in at least one of the proximal sleeve part 21 and the distal sleeve part 25, the flexibility of the protrusion parts 28 in the proximal sleeve part 21 and the distal sleeve part 25 can be made higher than the flexibility of the protrusion parts 28 in the proximal tapered part 22 and the distal tapered part 24. Therefore, even if the protrusion parts 28 in the proximal sleeve part 21 and the distal sleeve part 25, which are likely to come into contact with the wall of the vascular lumen when the balloon 2 advances or retreats through the vascular lumen, contact the vascular lumen wall, they are less likely to damage the vascular lumen wall.

In at least one of the proximal tapered part 22 and the distal tapered part 24, the ratio of the angle θ4 formed by a straight line Lc connecting the two outer layer ends 28bB and a straight line Ld connecting the outer layer end 28bB and the outer layer apex 28bT to the angle θ3 formed by a straight line La connecting the two inner layer ends 28aB and a straight line Lb connecting the inner layer end 28aB and the inner layer apex 28aT may be 1.10 times or more the ratio of the angle θ6 formed by a straight line Lc connecting the two outer layer ends 28bB and a straight line Ld connecting the outer layer end 28bB and the outer layer apex 28bT to the angle θ5 formed by a straight line La connecting the two inner layer ends 28aB and a straight line Lb connecting the inner layer end 28aB and the inner layer apex 28aT in at least one of the proximal sleeve part 21 and the distal sleeve part 25, 1.15 times or more, or 1.20 times or more. By setting the lower limit of the ratio between the ratio of the angle θ4 to the angle θ3 in at least one of the proximal tapered part 22 and the distal tapered part 24, and the ratio of the angle θ6 to the angle θ5 in at least one of the proximal sleeve part 21 and the distal sleeve part 25 within the above range, the protrusion parts 28 in the proximal sleeve part 21 and the distal sleeve part 25 can be made more flexible than the protrusion parts 28 in the proximal tapered part 22 and the distal tapered part 24, and the effect of making the protrusion parts 28 in the proximal sleeve part 21 and the distal sleeve part 25 less likely to damage the vascular lumen wall even when they come into contact with the vascular lumen wall can be enhanced. Additionally, in at least one of the proximal tapered part 22 and the distal tapered part 24, the ratio of the angle θ4 to the angle θ3 may be 5.0 times or less the ratio of the angle θ6 to the angle θ5 in at least one of the proximal sleeve part 21 and the distal sleeve part 25, 4.5 times or less, or 4.0 times or less. By setting the upper limit of the ratio between the ratio of the angle θ4 to the angle θ3 in at least one of the proximal tapered part 22 and the distal tapered part 24, and the ratio of the angle θ6 to the angle θ5 in at least one of the proximal sleeve part 21 and the distal sleeve part 25 within the above range, the rigidity of the protrusion parts 28 in the proximal tapered part 22 and the distal tapered part 24 can be increased, and the rigidity of the balloon 2 in the longitudinal axial direction x1 can be enhanced, thereby improving trackability.

As shown in FIGS. 2 and 3, an angle θa at the inner layer apex 28aT in a triangle formed by connecting the two inner layer ends 28aB and the inner layer apex 28aT in a cross-section perpendicular to the longitudinal direction x1 at the straight tubular part 23 may be greater than an angle θa at the inner layer apex 28aT in a triangle formed by connecting the two inner layer ends 28aB and the inner layer apex 28aT in a cross-section perpendicular to the longitudinal direction x1 at at least one of the proximal tapered part 22 and the distal tapered part 24. When the angle θa at the inner layer apex 28aT in the straight tubular part 23 is greater than the angle θa at the inner layer apex 28aT in at least one of the proximal tapered part 22 and the distal tapered part 24, the thickness of the outer layer 20b in the protrusion part 28 tends to be greater in the straight tubular part 23, whereby the rigidity of the protrusion part 28 increases and the protrusion part 28 can easily cut into a stenosis. In addition, in the proximal tapered part 22 and the distal tapered part 24, the thickness of the inner layer 20a in the protrusion part 28 tends to be greater, whereby the flexibility of the protrusion part 28 increases and the protrusion part 28 is less likely to damage the vascular lumen wall even when coming into contact with it.

An angle θa at the inner layer apex 28aT in a triangle formed by connecting the two inner layer ends 28aB and the inner layer apex 28aT in a cross-section perpendicular to the longitudinal direction x1 at the straight tubular part 23 may be 1.1 times or more, 1.2 times or more, or 1.3 times or more the angle θa at the inner layer apex 28aT in a triangle formed by connecting the two inner layer ends 28aB and the inner layer apex 28aT in a cross-section perpendicular to the longitudinal direction x1 at at least one of the proximal tapered part 22 and the distal tapered part 24. By setting the lower limit of the ratio of the angle θa at the inner layer apex 28aT in the straight tubular part 23 to the angle θa at the inner layer apex 28aT in at least one of the proximal tapered part 22 and the distal tapered part 24 within the above range, it is possible to increase the thickness of the outer layer 20b in the protrusion part 28 at the straight tubular part 23 compared to at least one of the proximal tapered part 22 and the distal tapered part 24, and thus increase the rigidity of the protrusion part 28. An angle θa at the inner layer apex 28aT in a triangle formed by connecting the two inner layer ends 28aB and the inner layer apex 28aT in a cross-section perpendicular to the longitudinal direction x1 at the straight tubular part 23 may be 5 times or less, 4 times or less, or 3 times or less the angle θa at the inner layer apex 28aT in a triangle formed by connecting the two inner layer ends 28aB and the inner layer apex 28aT in a cross-section perpendicular to the longitudinal direction x1 at at least one of the proximal tapered part 22 and the distal tapered part 24. By setting the upper limit of the ratio of the angle θa at the inner layer apex 28aT in the straight tubular part 23 to the angle θa at the inner layer apex 28aT in at least one of the proximal tapered part 22 and the distal tapered part 24 within the above range, it is possible to increase the thickness of the inner layer 20a in the protrusion part 28 at at least one of the proximal tapered part 22 and the distal tapered part 24 compared to the straight tubular part 23, and thereby improve the flexibility of the protrusion part 28.

An angle θa at the inner layer apex 28aT in a triangle formed by connecting the two inner layer ends 28aB and the inner layer apex 28aT in a cross-section perpendicular to the longitudinal direction x1 at at least one of the proximal tapered part 22 and the distal tapered part 24 may be 70 degrees or more, 80 degrees or more, or 90 degrees or more. By setting the lower limit of the angle θa at the inner layer apex 28aT at at least one of the proximal tapered part 22 and the distal tapered part 24 within the above range, the thickness of the inner layer 20a at the inner layer apex 28aT in the protrusion part 28 of the proximal tapered part 22 or the distal tapered part 24 can be increased, and the flexibility of the protrusion part 28 in the proximal tapered part 22 or the distal tapered part 24 can be enhanced. An angle θa at the inner layer apex 28aT in a triangle formed by connecting the two inner layer ends 28aB and the inner layer apex 28aT in a cross-section perpendicular to the longitudinal direction x1 at at least one of the proximal tapered part 22 and the distal tapered part 24 may be 160 degrees or less, 150 degrees or less, or 140 degrees or less. By setting the upper limit of the angle θa at the inner layer apex 28aT at at least one of the proximal tapered part 22 and the distal tapered part 24 within the above range, the inner layer 20a in the protrusion part 28 of the proximal tapered part 22 or the distal tapered part 24 is more likely to have a uniform thickness in the circumferential direction z1, and it becomes possible to reduce the likelihood of the inner layer 20a breaking during expansion of the balloon 2.

The angle θa at the inner layer apex 28aT in a triangle formed by connecting the two inner layer ends 28aB and the inner layer apex 28aT in a cross-section perpendicular to the longitudinal direction x1 at the straight tubular part 23 may be an obtuse angle, and the angle θa at the inner layer apex 28aT in a triangle formed by connecting the two inner layer ends 28aB and the inner layer apex 28aT in a cross-section perpendicular to the longitudinal direction x1 at at least one of the proximal tapered part 22 and the distal tapered part 24 may be an acute angle. That is the angle θa at the apex of a triangle formed by the two inner layer ends 28aB and the inner layer apex 28aT in a cross-section perpendicular to the longitudinal direction x1 at the straight tubular part 23 may be an angle of more than 90degrees and less than 180 degrees, and the angle θa at the inner layer apex 28aT in a triangle formed by the two inner layer ends 28aB and the inner layer apex 28aT at at least one of the proximal tapered part 22 and the distal tapered part 24 may be an angle of more than 0 degrees and less than 90 degrees. When the angle θa at the inner layer apex 28aT in the triangle formed by the two inner layer ends 28aB and the inner layer apex 28aT in the straight tubular part 23 is an obtuse angle, the thickness of the outer layer 20b in the protrusion part 28 of the straight tubular part 23 tends to be increased, thereby allowing the rigidity of the protrusion part 28 to be enhanced and improving its ability to cut into a stenosis. In addition, when the angle θa at the inner layer apex 28aT in the triangle formed by the two inner layer ends 28aB and the inner layer apex 28aT at at least one of the proximal tapered part 22 and the distal tapered part 24 is an acute angle, the thickness of the inner layer 20a in the protrusion part 28 of the proximal tapered part 22 or the distal tapered part 24 tends to be increased, thereby improving the flexibility of the protrusion part 28 and enhancing the effect of reducing the likelihood of damaging the vascular lumen wall.

As shown in FIGS. 2 and 3, the proportion of the area of the inner layer 20a in the protrusion part 28 in a cross-section perpendicular to the longitudinal direction x1 at the straight tubular part 23 may be smaller than the proportion of the area of the inner layer 20a in the protrusion part 28 in a cross-section perpendicular to the longitudinal direction x1 at at least one of the proximal tapered part 22 and the distal tapered part 24. That is, it may be preferable that the proportion of the inner layer 20a in the entire protrusion part 28 in the straight tubular part 23 is smaller than the proportion of the inner layer 20a in the entire protrusion part 28 at at least one of the proximal tapered part 22 and the distal tapered part 24. By making the proportion of the area of the inner layer 20a in the protrusion part 28 in the straight tubular part 23 smaller than the proportion of the area of the inner layer 20a in the protrusion part 28 at at least one of the proximal tapered part 22 and the distal tapered part 24, the proportion of the inner layer 20a in the protrusion part 28 becomes higher at the proximal tapered part 22 and the distal tapered part 24 than at the straight tubular part 23, thereby improving the flexibility of the protrusion part 28 at the proximal tapered part 22 and the distal tapered part 24. As a result, while maintaining the rigidity of the protrusion part 28 in the straight tubular part 23 to enhance the efficiency of incision of a stenosis, it is possible to make the protrusion part 28 more flexible at the proximal tapered part 22 and the distal tapered part 24, thereby reducing the likelihood of damaging the vascular lumen wall even when the protrusion part 28 comes into contact with it.

In a cross-section perpendicular to the longitudinal direction x1 at the straight tubular part 23, the proportion of the area of the inner layer 20a relative to the entire area of the protrusion part 28 may be 5% or more, 10% or more, or 15% or more. By setting the lower limit of the proportion of the area of the inner layer 20a in the protrusion part 28 in the straight tubular part 23 within the above range, a certain amount of the inner layer 20a is ensured to be present in the protrusion part 28 of the straight tubular part 23, and when the balloon 2 inflates in the circumferential direction z1, such as during expansion of the balloon 2, the inner layer 20a in the protrusion part 28 of the straight tubular part 23 becomes less likely to rupture. In addition, in a cross-section perpendicular to the longitudinal direction x1 at the straight tubular part 23, the proportion of the area of the inner layer 20a relative to the entire area of the protrusion part 28 may be 40% or less, 35% or less, or 30% or less. By setting the upper limit of the proportion of the area of the inner layer 20a in the protrusion part 28 in the straight tubular part 23 within the above range, the proportion of the outer layer 20b in the protrusion part 28 of the straight tubular part 23 can be increased, thereby making it possible to enhance the rigidity of the protrusion part 28 in the straight tubular part 23.

In a cross-section perpendicular to the longitudinal direction x1 at at least one of the proximal tapered part 22 and the distal tapered part 24, the proportion of the area of the inner layer 20a relative to the entire area of the protrusion part 28 may be 10% or more, 15% or more, or 20% or more. By setting the lower limit of the proportion of the area of the inner layer 20a in the protrusion part 28 at at least one of the proximal tapered part 22 and the distal tapered part 24 within the above range, the proportion of the inner layer 20a can be increased in the protrusion part 28 at the proximal tapered part 22 or the distal tapered part 24, thereby improving flexibility and reducing the likelihood of damaging the vascular lumen wall. In addition, in a cross-section perpendicular to the longitudinal direction x1 at at least one of the proximal tapered part 22 and the distal tapered part 24, the proportion of the area of the inner layer 20a relative to the entire area of the protrusion part 28 may be 50% or less, 45% or less, or 40% or less. By setting the upper limit of the proportion of the area of the inner layer 20a in the protrusion part 28 at at least one of the proximal tapered part 22 and the distal tapered part 24 within the above range, a certain amount of the outer layer 20b can be present in the protrusion part 28 at the proximal tapered part 22 or the distal tapered part 24, thereby enhancing the rigidity of the balloon 2 in the longitudinal direction x1 and improving the insertability into the vascular lumen.

As shown in FIGS. 2 and 4, in a cross-section perpendicular to the longitudinal direction x1 in the straight tubular part 23, the proportion of the area of the inner layer 20a in the protrusion part 28 may be smaller than the proportion of the area of the inner layer 20a in the protrusion part 28 in a cross-section perpendicular to the longitudinal direction x1 at at least one of the proximal sleeve part 21 and the distal sleeve part 25. That is, the proportion of the inner layer 20a within the entire protrusion part 28 in the straight tubular part 23 may be smaller than the proportion of the inner layer 20a within the entire protrusion part 28 at at least one of the proximal sleeve part 21 and the distal sleeve part 25. When the proportion of the area of the inner layer 20a in the protrusion part 28 in the straight tubular part 23 is smaller than the proportion of the area of the inner layer 20a in the protrusion part 28 at at least one of the proximal sleeve part 21 and the distal sleeve part 25, the proportion of the inner layer 20a in the protrusion part 28 at the proximal sleeve part 21 or the distal sleeve part 25 becomes higher than that in the straight tubular part 23, allowing the flexibility of the protrusion part 28 at the proximal sleeve part 21 or the distal sleeve part 25 to be improved. Accordingly, even if the protrusion part 28 at the proximal sleeve part 21 or the distal sleeve part 25 comes into contact with the vascular lumen wall, it is less likely to damage the wall, while the rigidity of the protrusion part 28 in the straight tubular part 23 can be maintained, allowing efficient incision of a stenosis.

In a cross-section perpendicular to the longitudinal direction x1 at at least one of the proximal sleeve part 21 and the distal sleeve part 25, the proportion of the area of the inner layer 20a to the entire area of the protrusion part 28 may be 20% or more, 25% or more, or 30% or more. By setting the lower limit of the proportion of the area of the inner layer 20a in the protrusion part 28 at at least one of the proximal sleeve part 21 and the distal sleeve part 25 within the above range, the proportion of the inner layer 20a in the protrusion part 28 at the proximal sleeve part 21 or the distal sleeve part 25 can be increased, thereby improving the flexibility and enhancing the effect of reducing the likelihood of damaging the vascular lumen wall. In addition, in a cross-section perpendicular to the longitudinal direction x1 at at least one of the proximal sleeve part 21 and the distal sleeve part 25, the proportion of the area of the inner layer 20a to the entire area of the protrusion part 28 may be 60% or less, 55% or less, or 50% or less. By setting the upper limit of the proportion of the area of the inner layer 20a in the protrusion part 28 at at least one of the proximal sleeve part 21 and the distal sleeve part 25 within the above range, the presence ratio of the outer layer 20b in the protrusion part 28 at the proximal sleeve part 21 or the distal sleeve part 25 can be ensured, and the rigidity of the balloon 2 in the longitudinal direction x1 can be enhanced, thereby improving the insertability into the vascular lumen.

As shown in FIGS. 3 and 4, the proportion of the area of the inner layer 20a to the entire area of the protrusion part 28 in a cross-section perpendicular to the longitudinal direction x1 at at least one of the proximal tapered part 22 and the distal tapered part 24 may be smaller than the proportion of the area of the inner layer 20a to the entire area of the protrusion part 28 in a cross-section perpendicular to the longitudinal direction x1 at at least one of the proximal sleeve part 21 and the distal sleeve part 25. In other words, the proportion of the inner layer 20a within the entire protrusion part 28 in at least one of the proximal tapered part 22 and the distal tapered part 24 may be smaller than the proportion of the inner layer 20a within the entire protrusion part 28 in at least one of the proximal sleeve part 21 and the distal sleeve part 25. By making the proportion of the area of the inner layer 20a in the protrusion part 28 in at least one of the proximal tapered part 22 and the distal tapered part 24 smaller than the proportion in the protrusion part 28 in the straight tubular part 23, the flexibility of the protrusion part 28 in the proximal sleeve part 21 or the distal sleeve part 25 can be increased in comparison with that in the proximal tapered part 22 or the distal tapered part 24. Accordingly, while maintaining the rigidity of the protrusion part 28 in the straight tubular part 23 and enhancing the efficiency of incising the stenosis, it becomes possible to prevent damage to the vessel lumen wall even when the protrusion part 28 of the proximal sleeve part 21 or the distal sleeve part 25 comes into contact with the vessel lumen wall.

### 2. Balloon Catheter

A first balloon catheter 1 according to one or more embodiments of the present invention includes the above-described first balloon 2 for a balloon catheter. A second balloon catheter 1 according to one or more embodiments of the present invention includes the above-described second balloon 2 for a balloon catheter. The balloon catheter 1 may include both the first balloon 2 for a balloon catheter and the second balloon 2 for a balloon catheter. As described in the section “1. Balloon for balloon catheter,” and as shown in FIG. 1, the balloon 2 is connected to a distal end portion of the shaft 30.

FIG. 1 illustrates a so-called rapid-exchange type balloon catheter 1, which has a guidewire port 50 located midway between the distal and proximal sides of the shaft 30 and has an inner shaft 60 that functions as a guidewire lumen from the guidewire port 50 to the distal side of the shaft 30. The balloon catheter 1 may have a distal shaft 31 and a proximal shaft 32, with the distal shaft 31 and the proximal shaft 32 being separate members. The proximal end part of the distal shaft 31 may be connected to the distal end part of the proximal shaft 32, thereby forming the shaft 30 that extends from the balloon 2 to the proximal end part of the balloon catheter 1. Alternatively, a single shaft 30 may extend from the balloon 2 to the proximal end part of the balloon catheter 1, or the distal shaft 31 and the proximal shaft 32 may each consist of multiple tube members.

The shaft 30 may have a fluid flow path and a guidewire lumen inside. To configure the shaft 30 to have the internal fluid flow path and the guidewire lumen, for example, the inner shaft 60 located inside the shaft 30 may function as the guidewire lumen, and the space between the shaft 30 and the inner shaft 60 may function as the fluid flow path. In such a configuration, the inner shaft 60 may extend from the distal end of the shaft 30 and passes through the balloon 2, the distal side of the balloon 2 may be connected to the inner shaft 60, and the proximal side of the balloon 2 may be connected to the shaft 30.

The shaft 30 may be composed of resin, metal, or a combination of resin and metal. By using resin as the material for the shaft, flexibility and elasticity can be more easily imparted to the shaft 30. By using metal as the material for the shaft 30, the delivering performance of the balloon catheter 1 can be improved. Examples of resin used for the shaft 30 include polyamide-based resin, polyester-based resin, polyurethane-based resin, polyolefin-based resin, fluorine-based resin, polyvinyl chloride-based resin, silicone-based resin, natural rubber, and synthetic rubber. Any one of these may be used alone, or two or more may be used in combination. Examples of metal used for the shaft 30, include stainless steel such as SUS 304 and SUS 316, platinum, nickel, cobalt, chromium, titanium, tungsten, gold, Ni—Ti alloys, Co—Cr alloys, or combinations thereof. When the shaft 30 is composed of the distal shaft 31 and proximal shaft 32 as separate members, the distal shaft 31 may be, for example, made of resin, and the proximal shaft 32 may be made of metal. The shaft 30 may also have a layered structure using different materials or the same material.

The balloon 2 and the shaft 30 may be joined by adhesive bonding, welding, or by attaching a ring-shaped member at the point where the end of the balloon 2 and the shaft 30 overlap to swage them. Of these, the balloon 2 and the shaft 30 may be joined by welding. By welding the balloon 2 and the shaft 30, the bond between the balloon 2 and the shaft 30 is difficult to be released even when the balloon 2 is repeatedly inflated and deflated, easily increasing the strength of the bond between them.

The balloon catheter 1 may be provided with a tip member 70 at its distal end part. The tip member 70 may be provided at the distal end part of the balloon catheter 1 by being connected to the distal end part of the balloon 2 as a separate component from the inner shaft 60, or the inner shaft 60 extending distally beyond the distal end of the balloon 2 may function as the tip member 70.

A radiopaque marker 80 may be placed on the inner shaft 60 inside the balloon 2 at the location of the balloon 2 in the longitudinal axis direction x1, so that the position of the balloon 2 can be confirmed radiographically. The radiopaque marker 80 may be placed at a position corresponding to both ends of the straight tubular part 23 of the balloon 2, or may be placed at a position corresponding to the center of the straight tubular part 23 in the longitudinal axis direction x1.

A hub 5 may be provided at a proximal side of the shaft 30, and the hub 5 may be provided with a fluid inlet 6 that is connected to the flow channel of the fluid supplied to the interior of the balloon 2.

The shaft 30 and the hub 5 may be joined by, for example, adhesive bonding or welding. Of these, the shaft 30 and the hub 5 may be joined by adhesive bonding. The adhesive bonding of the shaft 30 and hub 5 can increase the bonding strength of the shaft 30 and hub 5 to increase durability of the balloon catheter 1 when the materials forming the shaft 30 and hub 5 are different, for example, in a case where the shaft 30 is made of material having high flexibility and the hub 5 is made of material having high stiffness. Although not shown in the figures, one or more embodiments of the present invention are also applicable to a so-called over-the-wire type balloon catheter that has a guidewire lumen extending from the distal end to the proximal end of the shaft. In the case of the over-the-wire type, the inflation lumen and the guidewire lumen may extend to a hub positioned at the proximal side, and the proximal openings of each lumen may be provided in the hub having a bifurcated structure.

In the case of the rapid-exchange type catheter, the outer wall of the distal shaft 31 and/or the proximal shaft 32 may be coated as appropriate, or both the distal shaft 31 and the proximal shaft 32 may be coated. In the case of the over-the-wire type catheter, the outer wall of the outer shaft may be coated as appropriate.

The coating can be a hydrophilic or hydrophobic coating, depending on the purpose, and can be applied by dipping the shaft 30 into a hydrophilic or hydrophobic coating agent, applying a hydrophilic or hydrophobic coating agent to the outer wall of the shaft 30, or coating the outer wall of the shaft 30 with a hydrophilic or hydrophobic coating agent. The coating agent may contain medical agents and additives.

Hydrophilic coating agents include hydrophilic polymers such as polyvinyl alcohol, polyethylene glycol, polyacrylamide, polyvinyl pyrrolidone, methyl vinyl ether maleic anhydride copolymer, and hydrophilic coating agents made of any combination thereof.

Hydrophobic coating agents include polytetrafluoroethylene (PTFE), ethylene-propylene fluoride (FEP), perfluoroalkoxy alkane (PFA), silicone oil, hydrophobic urethane resin, carbon coat, diamond coat, diamond-like carbon (DLC) coating, ceramic coating, and substances with low surface free energy terminated with an alkyl group or a perfluoroalkyl group.

### 3. Method for Producing Balloon Catheter

A method for producing the first and second balloon catheters according to one or more embodiments of the present invention is a method for producing the first and second balloon catheters described above, the method has a step of preparing a parison having a radial direction, a circumferential direction, and a longitudinal axis direction, the parison having a lumen extending in the longitudinal axis direction, and a step of stretching the parison to produce a balloon including the proximal sleeve part, the proximal tapered part, the straight tubular part, the distal tapered part, and the distal sleeve part, the balloon including the protrusion part protruding outward in the radial direction and extending in the longitudinal axis direction, wherein the parison includes an outer layer, and an inner layer composed of a material having a Shore D hardness lower than that of the outer layer; and a protruding region including the protrusion part that protrudes outward in the radial direction and extends in the longitudinal axis direction, and a non-protruding region other than the protruding region, wherein, in a cross section perpendicular to the longitudinal axis direction, the inner layer includes a thin portion in the non-protruding region and a thick portion in the protruding region, the thick portion having a thickness greater than a thickness of the thin portion.

First, the first balloon for a balloon catheter will be described. In the method according to one or more embodiments of the present invention, a parison has an outer layer and an inner layer composed of a material having a Shore D hardness lower than that of the outer layer, and includes a protruding region and a non-protruding region. In a cross-section perpendicular to the longitudinal direction, the inner layer has a thin portion in the non-protruding region and a thick portion in the protruding region. By producing a balloon through stretching such a parison, it is possible to produce the “2. Balloon catheter” having the “1. Balloon for balloon catheter” in which, in the straight tubular part, the angle formed in the first direction of the circumferential direction by the straight line connecting two inner layer ends and the straight line connecting the inner layer end and the inner layer apex is smaller than the angle formed in the first direction of the circumferential direction by the straight line connecting two outer layer ends and the straight line connecting the outer layer end and the outer layer apex.

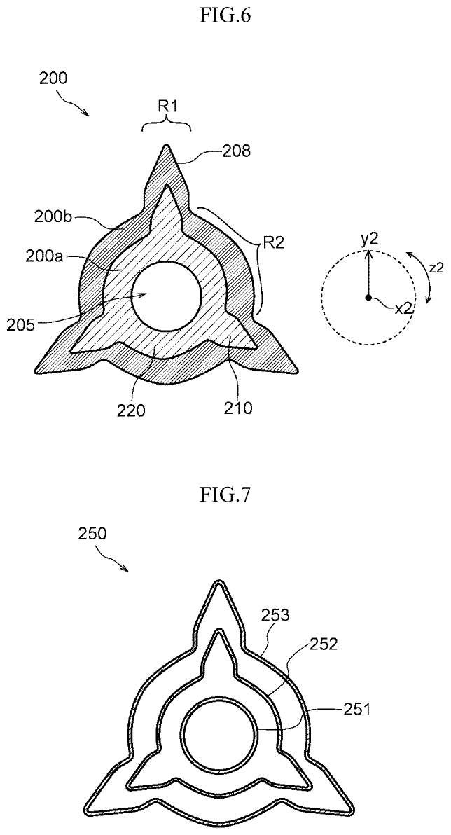

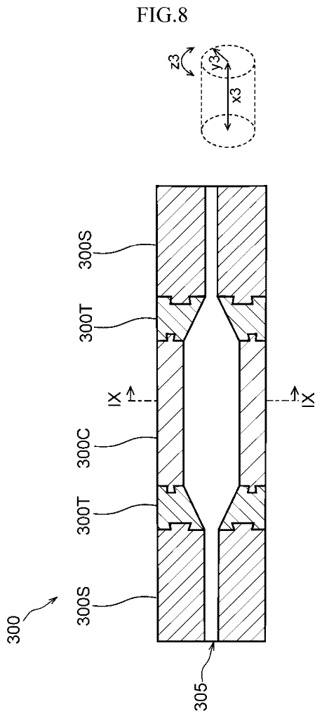

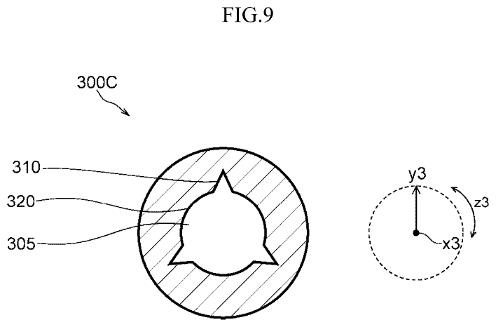

A method for producing a balloon catheter according to one or more embodiments of the present invention will be described with reference to FIGS. 5 to 9. FIG. 5 is a perspective view of a parison before stretching according to one or more embodiments of the present invention. FIG. 6 is a cross-sectional view taken along line VI-VI of the parison shown in FIG. 5, and FIG. 7 is a cross-sectional view in a direction perpendicular to the longitudinal direction of a parison mold used to produce the parison shown in FIG. 6. FIG. 8 is a longitudinal cross-sectional view of a mold used for stretching the parison in the method according to one or more embodiments of the present invention. FIG. 9 is a cross-sectional view taken along line IX-IX of the mold shown in FIG. 8.

First, a parison 200 is prepared. As shown in FIG. 5, the parison 200 is a tubular member composed of a resin and has a lumen 205. The parison 200 has a first end 201 and a second end 202, and extends in a longitudinal direction x2 from the first end 201 toward the second end 202. The parison 200 also has a radial direction y2 and a circumferential direction z2, similar to the balloon 2.

As shown in FIG. 6, the parison 200 includes an outer layer 200b and an inner layer 200a composed of a material having a Shore D hardness lower than that of the outer layer 200b. The materials constituting the inner layer 200a and the outer layer 200b, as well as their Shore D hardness, can be referred to in the descriptions of the inner layer 20a and the outer layer 20b provided in the section “1. Balloon for balloon catheter,” including the resins forming those layers and their respective Shore D hardness.

The parison 200 includes a protruding region R1 including a protrusion part 208 that protrudes outward in the radial direction y2 and extends in the longitudinal direction x2, and a non-protruding region R2 other than the protruding region R1. By stretching the parison 200, the protrusion part 208 can be molded into the protrusion part 28 of the balloon 2, and the portion of the non-protruding region R2 can be molded into the balloon body part 20 other than the protrusion part 28.

As shown in FIG. 6, a plurality of protrusion parts 208 may be provided in the circumferential direction z2. Although not illustrated, a single protrusion part 208 may also be provided in the circumferential direction z2. When a plurality of protrusion parts 208 are provided in the circumferential direction z2, the plurality of protrusion parts 208 may be spaced apart from one another in the circumferential direction z2, or may be arranged at equal intervals in the circumferential direction z2.

As shown in FIG. 6, in a cross-section perpendicular to the longitudinal direction x2, the inner layer 200a includes a thin portion 220 in the non-protruding region R2, and a thick portion 210 in the protruding region R1, the thick portion 210 having a thickness greater than that of the thin portion 220. By providing the thick portion 210 in the inner layer 200a in the protruding region R1, it becomes possible to produce a balloon 2 in which the angle θ1 formed in the first direction d1 of the circumferential direction z1 by a straight line La connecting the two inner layer ends 28aB and a straight line Lb connecting the inner layer end 28aB and the inner layer apex 28aT at the straight tubular part 23 is smaller than the angle θ2 formed in the first direction d1 of the circumferential direction z1 by a straight line Lc connecting the two outer layer ends 28bB and a straight line Ld connecting the outer layer end 28bB and the outer layer apex 28bT.

A parison 200 as described above can be produced, for example, by extruding a resin using a parison mold 250 as shown in FIG. 7. As shown in FIG. 7, the parison mold 250 includes a first cylindrical member 251, a second cylindrical member 252, and a third cylindrical member 253. The first cylindrical member 251 may have a cylindrical shape so as to form the lumen 205 of the parison 200. The second cylindrical member 252 may have a cylindrical shape with protrusions so as to form the thick portion 210 and the thin portion 220 of the inner layer 200a. The third cylindrical member 253 may have a cylindrical shape with protrusions so as to form the protrusions 208. The parison 200 including the protrusions 208, the inner layer 200a, and the outer layer 200b, and in which the inner layer 200a has the thick portion 210 in the protruding region R1, can be produced by introducing resin for forming the inner layer 200a into the space between the outer surface of the first cylindrical member 251 and the inner surface of the second cylindrical member 252, and by introducing resin for forming the outer layer 200b into the space between the outer surface of the second cylindrical member 252 and the inner surface of the third cylindrical member 253, followed by extrusion molding.

The material constituting the parison mold 250 may be metal, and the metal may be iron, copper, aluminum, or an alloy thereof. Examples of iron alloys include stainless steel, examples of copper alloys include brass, and examples of aluminum alloys include duralumin. From the perspective of having sufficient electrical conductivity and strength as well as ease of processing, it may be preferable that the parison mold 250 is made of stainless steel.

By stretching the parison 200, the balloon 2 having the proximal sleeve part 21, the proximal tapered part 22, the straight tubular part 23, the distal tapered part 24, the distal sleeve part 25, and the protrusion part 28 is produced. In this process, a mold 300 as shown in FIG. 8 can be used. The mold 300 has a longitudinal direction x3, a radial direction y3, and a circumferential direction z3, extends in the longitudinal direction x3, and has a lumen 305 into which the parison 200 is inserted. A portion of the parison 200 in the longitudinal direction x2 may be disposed within the lumen 305 of the mold 300.

The parison 200 may be biaxially stretched. The balloon 2 may be formed by biaxial stretch blow molding of the parison 200. In the production of the balloon 2, by biaxially stretching the parison 200, the parison 200 can be uniformly stretched, thereby enabling the production of the balloon 2 with high overall strength and stable quality.

The mold 300 may include, in the longitudinal direction x3, a mold straight tubular part 300C that forms the straight tubular part of the balloon 2, two mold tapered parts 300T disposed on both sides of the mold straight tubular part 300C and forming the tapered parts of the balloon 2, and two mold sleeve parts 300S disposed farther from the mold straight tubular part 300C than the mold tapered parts 300T and forming the sleeve parts of the balloon 2. With this configuration, the straight tubular part 23 of the balloon 2 can be formed by the mold straight tubular part 300C, the proximal tapered part 22 and distal tapered part 24 can be formed by the mold tapered parts 300T, and the proximal sleeve part 21 and distal sleeve part 25 can be formed by the mold sleeve parts 300S.

The mold 300 may be composed of a single member or may be composed of a plurality of members. As shown in FIG. 8, it may be configured by connecting a plurality of mold members to each other in the longitudinal direction x3. For example, the mold straight tubular part 300C, the mold tapered part 300T, and the mold sleeve part 300S may be separate mold members and may be connected to each other in the longitudinal direction x3. The mold 300 may also be dividable in the radial direction y. This configuration facilitates insertion of the parison 200 into the inner cavity 305 of the mold 300. As shown in FIG. 8, each mold member may be joined to an adjacent mold member by engagement, or, although not shown, magnets may be attached to each of the adjacent mold members and joined by magnetic attraction.

As shown in FIG. 9, the inner cavity 305 of the mold 300 may be formed of a groove 310 that is recessed outward in the radial direction y3 and extends in the longitudinal direction x3, and a cylindrical wall part 320 other than the groove 310. This configuration allows the protruding part 208 of the parison 200 to enter the groove 310, thereby forming the protrusion part 28 of the balloon 2. A plurality of grooves 310 may be provided in the circumferential direction z3, or, although not shown, only one groove 310 may be provided in the circumferential direction z3. When a plurality of grooves 310 are provided in the circumferential direction z3, the grooves 310 may be spaced apart in the circumferential direction z3, or may be arranged at equal intervals in the circumferential direction z3.

The groove 310 may be provided in the mold straight tubular part 300C, but may also be provided in the mold tapered part 300T or the mold sleeve part 300S. By providing the groove 310 in the mold straight tubular part 300C, the protrusion part 28 can be formed in the straight tubular part 23 of the balloon 2, thereby improving the efficiency of incision of a stenosis by the balloon 2. The depth of the groove 310 provided in the mold tapered part 300T or the mold sleeve part 300S may be shallower than, or equal to, the depth of the groove 310 provided in the mold straight tubular part 300C.

The material constituting the mold 300 may be a metal, and the metal may be iron, copper, aluminum, or an alloy thereof. For example, examples of iron alloys include stainless steel; examples of copper alloys include brass; and examples of aluminum alloys include duralumin. From the viewpoint of having sufficient electrical conductivity and strength, and ease of processing, the mold 300 may be made of stainless steel.

Next, a method for producing the second balloon catheter will be described. In the following description of the method for producing the second balloon catheter, descriptions that overlap with those of the method for producing the first balloon catheter described above will be omitted.

In the method according to the present embodiment, a parison includes an outer layer and an inner layer made of a material having a lower Shore D hardness than the outer layer, and includes a protruding region and a non-protruding region. In a cross-section perpendicular to the longitudinal direction, the inner layer includes a thin portion in the non-protruding region and a thick portion having a greater thickness than the thin portion in the protruding region. By producing a balloon through biaxial stretching of such a parison, it is possible to manufacture the “2. Balloon catheter” including the second “1. Balloon for balloon catheter,” in which the ratio of the angle θ2 formed in the first direction of the circumferential direction by a straight line connecting two outer layer ends and a straight line connecting an outer layer end and an outer layer apex in the straight tubular part to the angle θ1 formed in the first direction of the circumferential direction by a straight line connecting two inner layer ends and a straight line connecting an inner layer end and an inner layer apex in the straight tubular part is greater than the ratio of the angle θ4 formed in the first direction of the circumferential direction by a straight line connecting two outer layer ends and a straight line connecting an outer layer end and an outer layer apex in at least one of the proximal tapered part and the distal tapered part to the angle θ3 formed in the first direction of the circumferential direction by a straight line connecting two inner layer ends and a straight line connecting an inner layer end and an inner layer apex in the same part.

As shown in FIG. 6, in a cross-section perpendicular to the longitudinal direction x2, the inner layer 200a has the thin portion 220 in the non-protruding region R2, and has the thick portion 210 having a thickness greater than the thickness of the thin portion 220 in the protruding region R1. By the inner layer 200a having the thick portion 210 in the protruding region R1, it becomes possible to produce the balloon 2 in which the ratio of the angle θ2 formed by the straight line Lc connecting the two outer layer base ends 28bB and the straight line Ld connecting the outer layer base end 28bB and the outer layer apex 28bT in the first direction d1 of the circumferential direction z1 to the angle θ1 formed by the straight line La connecting the two inner layer base ends 28aB and the straight line Lb connecting the inner layer base end 28aB and the inner layer apex 28aT in the first direction d1 of the circumferential direction z1 in the straight tubular part 23 is greater than the ratio of the angle θ4 formed by the straight line Lc and the straight line Ld in the first direction d1 of the circumferential direction z1 to the angle θ3 formed by the straight line La and the straight line Lb in the first direction d1 of the circumferential direction z1 in at least one of the proximal tapered part 22 and the distal tapered part 24.

Such a parison 200 can be manufactured by extrusion-molding a resin using the parison mold 250 shown in FIG. 7 described above.

The present application claims priority based on Japanese Patent Application No. 2022-183541 filed on Nov. 16, 2022 and Japanese Patent Application No. 2022-183543 filed on Nov. 16, 2022. All the contents described in Japanese Patent Application No. 2022-183541 filed on Nov. 16, 2022 and Japanese Patent Application No. 2022-183543 filed on Nov. 16, 2022 are incorporated herein by reference.

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims.

### DESCRIPTION OF REFERENCE SIGNS

1: balloon catheter
2: balloon for balloon catheter
5: hub
6: fluid inlet
20: balloon body part
20a: inner layer
20b: outer layer
21: proximal sleeve part
22: proximal tapered part
23: straight tubular part
24: distal tapered part
25: distal sleeve part
28: protrusion part
28T: apex
28B: base end
28a: inner layer protrusion part
28aT: inner layer apex
28aB: inner layer end
28b: outer layer protrusion part
28bT: outer layer apex
28bB: outer layer end
30: shaft
31: distal shaft
32: proximal shaft
50: guidewire port
60: inner shaft
70: tip member
80: radiopaque marker
200: parison
200a: inner layer of parison
200b: outer layer of parison
201: first end of parison
202: second end of parison
205: lumen of parison
208: protrusion part of parison
210: thick portion
220: thin portion
250: parison mold
251: first cylindrical member
252: second cylindrical member
253: third cylindrical member
300: mold
300C: mold straight tubular part
300S: mold sleeve part
300T: mold tapered part
305: lumen of mold
310: groove
320: cylindrical wall part
Lp: straight line connecting midpoint of base ends and apex
Lv: perpendicular line of base end
La: straight line connecting two inner layer ends
Lb: straight line connecting inner layer end and inner layer apex
Lc: straight line connecting two outer layer ends
Ld: straight line connecting outer layer end and outer layer apex
θ1: angle formed, in a cross-section perpendicular to the longitudinal direction in the straight tubular part, between the straight line La and straight line Lb in the first direction of the circumferential direction
θ2: angle formed, in a cross-section perpendicular to the longitudinal direction in the straight tubular part, between the straight line Lc and straight line Ld in the first direction of the circumferential direction
θ3: angle formed, in a cross-section perpendicular to the longitudinal direction in at least one of the proximal tapered part and distal tapered part, between the straight line La and straight line Lb in the first direction of the circumferential direction
θ4: angle formed, in a cross-section perpendicular to the longitudinal direction in at least one of the proximal tapered part and distal tapered part, between the straight line Lc and straight line Ld in the first direction of the circumferential direction
θ5: angle formed, in a cross-section perpendicular to the longitudinal direction in at least one of the proximal sleeve part and distal sleeve part, between the straight line La and straight line Lb in the first direction of the circumferential direction
θ6: angle formed, in a cross-section perpendicular to the longitudinal direction in at least one of the proximal sleeve part and distal sleeve part, between the straight line Lc and straight line Ld in the first direction of the circumferential direction

θa: angle at the inner layer apex in a triangle formed by connecting the two inner layer ends and inner layer apex
θb: angle at the outer layer apex in a triangle formed by connecting the two outer layer ends and outer layer apex

1. A balloon for a balloon catheter having a longitudinal axis direction, a radial direction, and a circumferential direction, comprising:
an outer layer;
an inner layer composed of a material having a Shore D hardness lower than that of the outer layer; and
a protrusion part that protrudes outward in the radial direction and extends in the longitudinal axis direction, wherein:
the balloon comprises: a straight tubular part; a proximal tapered part located proximal to the straight tubular part; a proximal sleeve part located proximal to the proximal tapered part; a distal tapered part located distal to the straight tubular part; and a distal sleeve part located distal to the distal tapered part; and
in a cross section perpendicular to the longitudinal axis direction at the straight tubular part, a region where the protrusion part is present includes:
an outer layer protrusion part formed by the outer layer and protruding outward in the radial direction; and
an inner layer protrusion part formed by the inner layer and protruding outward in the radial direction, wherein:
the outer layer protrusion part includes: an outer layer apex being an apex of the outer layer protrusion part; and outer layer ends located on both sides in the circumferential direction at respective circumferential ends of the outer layer protrusion part;
the inner layer protrusion part includes: an inner layer apex being an apex of the inner layer protrusion part; and inner layer ends located on both sides in the circumferential direction at respective circumferential ends of the inner layer protrusion part; and
in the cross section perpendicular to the longitudinal axis direction at the straight tubular part, an angle formed in a first direction in the circumferential direction between a straight line that connects the two inner layer ends and a straight line that connects the inner layer apex and one of the two inner layer ends is smaller than an angle formed in the first direction in the circumferential direction between a straight line that connects the two outer layer ends and a straight line that connects the outer layer apex and one of the two outer layer ends.

2. The balloon for the balloon catheter according to claim 1, wherein, in a cross section perpendicular to the longitudinal axis direction at the straight tubular part, the inner layer apex is located further outward in the radial direction than a straight line that connects the two outer layer ends.

3. The balloon for the balloon catheter according to claim 1, wherein:
in a cross section perpendicular to the longitudinal axis direction at the straight tubular part, an angle at the inner layer apex in a triangle formed by connecting the two inner layer ends and the inner layer apex is an obtuse angle; and
in the cross section perpendicular to the longitudinal axis direction at the straight tubular part, an angle at the outer layer apex in a triangle formed by connecting the two outer layer ends and the outer layer apex is an acute angle.

4. The balloon for the balloon catheter according to claim 1, wherein, in a cross section perpendicular to the longitudinal axis direction at the straight tubular part, an area of the inner layer protrusion part is smaller than an area of the outer layer protrusion part.

5. The balloon for the balloon catheter according to claim 1, wherein, in a cross section perpendicular to the longitudinal axis direction at at least one of the proximal tapered part and the distal tapered part, an angle formed in the first direction in the circumferential direction between a straight line that connects the two inner layer ends and a straight line that connects the inner layer apex and one of the two inner layer ends is smaller than an angle formed in the first direction in the circumferential direction between a straight line that connects the two outer layer ends and a straight line that connects the outer layer apex and one of the two outer layer ends.

6. The balloon for the balloon catheter according to claim 1, wherein, in a cross section perpendicular to the longitudinal axis direction at at least one of the proximal sleeve part and the distal sleeve part, an angle formed in the first direction in the circumferential direction between a straight line that connects the two inner layer ends and a straight line that connects the inner layer apex and one of the two inner layer ends is greater than an angle formed in the first direction in the circumferential direction between a straight line that connects the two outer layer ends and a straight line that connects the outer layer apex and one of the two outer layer ends.

7. The balloon for the balloon catheter according to claim 1, wherein, in a cross section perpendicular to the longitudinal axis direction at at least one of the proximal sleeve part and the distal sleeve part, an angle formed in the first direction in the circumferential direction between a straight line that connects the two inner layer ends and a straight line that connects the inner layer apex and one of the two inner layer ends is smaller than an angle formed in the first direction in the circumferential direction between a straight line that connects the two outer layer ends and a straight line that connects the outer layer apex and one of the two outer layer ends.

8. A balloon catheter comprising the balloon according to claim 1.

9. A method for producing the balloon catheter according to claim 8, the method comprising:
preparing a parison having a radial direction, a circumferential direction, and a longitudinal axis direction, the parison having a lumen extending in the longitudinal axis direction; and
stretching the parison to produce a balloon including the proximal sleeve part, the proximal tapered part, the straight tubular part, the distal tapered part, and the distal sleeve part, the balloon including the protrusion part protruding outward in the radial direction and extending in the longitudinal axis direction, wherein the parison includes:
an outer layer, and an inner layer composed of a material having a Shore D hardness lower than that of the outer layer; and
a protruding region including the protrusion part that protrudes outward in the radial direction and extends in the longitudinal axis direction, and a non-protruding region other than the protruding region, wherein:
in a cross section perpendicular to the longitudinal axis direction, the inner layer includes a thin portion in the non-protruding region and a thick portion in the protruding region, the thick portion having a thickness greater than a thickness of the thin portion.

10. A balloon for a balloon catheter having a longitudinal axis direction, a radial direction, and a circumferential direction, comprising:
an outer layer;
an inner layer composed of a material having a Shore D hardness lower than that of the outer layer; and
a protrusion part that protrudes outward in the radial direction and extends in the longitudinal axis direction, wherein:
the balloon comprises: a straight tubular part; a proximal tapered part located proximal to the straight tubular part; a proximal sleeve part located proximal to the proximal tapered part; a distal tapered part located distal to the straight tubular part; and a distal sleeve part located distal to the distal tapered part; and
in a cross section perpendicular to the longitudinal axis direction at the straight tubular part, a region where the protrusion part is present includes:
an outer layer protrusion part formed by the outer layer and protruding outward in the radial direction; and
an inner layer protrusion part formed by the inner layer and protruding outward in the radial direction, wherein:
the outer layer protrusion part includes: an outer layer apex being an apex of the outer layer protrusion part; and outer layer ends located on both sides in the circumferential direction at respective circumferential ends of the outer layer protrusion part;
the inner layer protrusion part includes: an inner layer apex being an apex of the inner layer protrusion part; and inner layer ends located on both sides in the circumferential direction at respective circumferential ends of the inner layer protrusion part; and
a ratio (θ2/θ1) of an angle θ2 formed in a first direction in the circumferential direction between a straight line that connects the two outer layer ends and a straight line that connects the outer layer apex and one of the two outer layer ends to an angle θ1 formed in the first direction in the circumferential direction between a straight line that connects the two inner layer ends and a straight line that connects the inner layer apex and one of the two inner layer ends, in the cross section perpendicular to the longitudinal axis direction at the straight tubular part, is greater than a ratio (θ4/θ3) of an angle θ4 formed in the first direction in the circumferential direction between a straight line that connects the two outer layer ends and a straight line that connects the outer layer apex and one of the two outer layer ends to an angle θ3 formed in the first direction in the circumferential direction between a straight line that connects the two inner layer ends and a straight line that connects the inner layer apex and one of the two inner layer ends, in a cross section perpendicular to the longitudinal axis direction at at least one of the proximal tapered part and the distal tapered part.

11. The balloon for the balloon catheter according to claim 10, wherein the ratio (θ2/θ1) is greater than a ratio (θ6/θ5) of an angle θ6 formed in the first direction in the circumferential direction between a straight line that connects the two outer layer ends and a straight line that connects the outer layer apex and one of the two outer layer ends to an angle θ5 formed in the first direction in the circumferential direction between a straight line that connects the two inner layer ends and a straight line that connects the inner layer apex and one of the two inner layer ends, in a cross section perpendicular to the longitudinal axis direction at at least one of the proximal sleeve part and the distal sleeve part.

12. The balloon for the balloon catheter according to claim 10, wherein the ratio (θ4/θ3) is greater than a ratio (θ6/θ5) of an angle θ6 formed in the first direction in the circumferential direction between a straight line that connects the two outer layer ends and a straight line that connects the outer layer apex and one of the two outer layer ends to an angle θ5 formed in the first direction in the circumferential direction between a straight line that connects the two inner layer ends and a straight line that connects the inner layer apex and one of the two inner layer ends, in a cross section perpendicular to the longitudinal axis direction at at least one of the proximal sleeve part and the distal sleeve part.

13. The balloon for the balloon catheter according to claim 10, wherein an angle at the inner layer apex formed in a triangle defined by connecting the two inner layer ends and the inner layer apex, in a cross section perpendicular to the longitudinal axis direction at the straight tubular part, is greater than an angle at the inner layer apex formed in a triangle defined by connecting the two inner layer ends and the inner layer apex, in a cross section perpendicular to the longitudinal axis direction at at least one of the proximal tapered part and the distal tapered part.

14. The balloon for the balloon catheter according to claim 10, wherein a proportion of an area of the inner layer in the protrusion part in a cross section perpendicular to the longitudinal axis direction at the straight tubular part is smaller than a proportion of an area of the inner layer in the protrusion part in a cross section perpendicular to the longitudinal axis direction at at least one of the proximal tapered part and the distal tapered part.

15. A balloon catheter comprising the balloon according to claim 10.

16. A method for producing the balloon catheter according to claim 15, the method comprising:
preparing a parison having a radial direction, a circumferential direction, and a longitudinal axis direction, the parison having a lumen extending in the longitudinal axis direction; and
stretching the parison to produce a balloon including the proximal sleeve part, the proximal tapered part, the straight tubular part, the distal tapered part, and the distal sleeve part, the balloon including the protrusion part protruding outward in the radial direction and extending in the longitudinal axis direction, wherein the parison includes:
an outer layer, and an inner layer composed of a material having a Shore D hardness lower than that of the outer layer; and
a protruding region including the protrusion part that protrudes outward in the radial direction and extends in the longitudinal axis direction, and a non-protruding region other than the protruding region, wherein:
in a cross section perpendicular to the longitudinal axis direction, the inner layer includes a thin portion in the non-protruding region and a thick portion in the protruding region, the thick portion having a thickness greater than a thickness of the thin portion.
